(19) United States
(12) Patent Application Publication
(10) Pub. No.: US 2005/0276798 A1
(43) Pub. Date: Dec. 15, 2005

(54) CD4-IgG2 formulations

(76) Inventors: William C. Olson, Ossining, NY (US); Kesh Prakash, Holland, PA (US); Norbert Schulke, Dedham, MA (US)

Correspondence Address: COOPER & DUNHAM, LLP, 1185 AVENUE OF THE AMERICAS, NEW YORK, NY, 10036, US

(21) Appl. No.: 11/086,611

(22) Filed: Mar. 21, 2005

Related U.S. Application Data
(60) Provisional application No. 60/554,883, filed on Mar. 19, 2004.

(51) Int. Cl. A61K 39/395; A61K 31/7076; A61K 31/7072; A61K 31/551; A61K 39/42; A61K 31/522

(52) U.S. Cl. 424/133.1; 514/220; 514/263.31; 514/47; 514/50; 514/53

(57) ABSTRACT

This invention provides formulations of the anti-HIV therapeutic, CD4-IgG2, that contain higher concentrations of the therapeutic than were previously prepared, are stable, and safe to administer. Methods for making high-concentration CD4-IgG2 formulations by first concentrating CD4-IgG2 to about 50 mg/ml in a buffer comprising about 6.7 mM histidine/2% maltose, pH 6.0, then lyophilizing the sample and reconstituting it to about 150 mg/ml in a buffer comprising about 20 mM histidine/6% maltose, pH 6.0, are provided. Such high-concentration CD4-IgG2 formulations are suitable for intravenous, subcutaneous and intramuscular delivery, the latter two routes being potentially useful for facilitating self-administration by HIV-infected individuals. This invention is also directed to methods of using the CD4-IgG2 formulations to inhibit or prevent infect CD4+ cells from becoming infected with HIV, and to treat subjects having CD4+ cells infected with HIV.

Throughout this application, various patents and publications are referenced in parentheses by, respectively, the inventors' name and patent number and the author name and date. Full citations for these patents and publications may be found at the end of the specification immediately preceding the claims. The disclosures of these patents and publications in their entireties are hereby incorporated by reference into this application in order to more fully describe the state of the art as known to those skilled therein as of the date of the invention described and claimed herein. However, the citation of a reference herein should not be construed as an acknowledgement that such reference is prior art to the present invention.

### FIELD OF THE INVENTION

The present invention relates to stable, liquid and lyophilized formulations of a CD4-IgG2 chimeric heterotetramer useful in prevention and treatment of HIV-1 infections. More specifically the invention relates to highly concentrated, stable formulations of the CD4-IgG2 chimeric heterotetramer that allows for parenteral administration.

### BACKGROUND OF THE INVENTION

The human immunodeficiency virus type 1 (HIV-1) which causes human acquired immunodeficiency syndrome (AIDS) infects primarily helper T lymphocytes and monocytes/macrophages, cells that express surface CD4 receptor protein. This selectivity occurs because the initial phase of the HIV-1 replicative cycle requires a high-affinity interaction between CD4 on the surface of susceptible cells and the glycoprotein, gp120, on the exterior envelope of HIV (Lasky et al., 1987). Consequently, molecules that incorporate the gp-120 binding region of CD4 have the potential to bind and neutralize all variants of HIV-1. This has prompted the development of CD4-based therapeutic agents, including soluble CD4 (sCD4; Deen et al., 1988; Fisher et al., 1988) and CD4-immunoglobulin fusion proteins (Traunecker et al., 1989; Capon et al., 1989; Byrn et al., 1990; Allaway et al., 1993; Allaway et al., 1995). These therapeutic molecules neutralize HIV-1 and prevent its entry into human cells by various mechanisms including competitively inhibiting attachment of the virus to susceptible cells, dissociating gp12O from the viral surface, and inhibiting intercellular transmission of the virus initiated by virus-mediated cell fusion (Klatzmann et al., 1990; Moore et al., 1990).

CD4-IgG2 (Allaway et al., 1995), also referred to herein as CD4-IgG2 chimeric heterotetramer or PRO 542, is an HIV-1 attachment and entry inhibitor manufactured by Progenics Pharmaceuticals, Inc. of Tarrytown, N.Y., that is currently undergoing large-scale Phase II clinical trials as an anti-HIV therapeutic (Progenics, 2002). CD4-IgG2 is a novel chimeric protein in which polypeptides comprising both the heavy and light chain constant regions of human IgG2 have been fused to the V1 and V2 gp120-binding domains of human CD4 (see FIG. 1; Allaway et al., 1995; U.S. Pat. No. 6,187,748 B1 to Maddon and Beaudry, 2001). This fusion protein, which is assembled and secreted as a heterotetrameric molecule, contains two IgG2-CD4 chimeric heavy chains and two CD4-kappa chimeric light chains. The heavy chains are encoded by the expression vector designated CD4-IgG2HC-pRcCMV (ATCC No. 75193), and the light chains are encoded by the expression vector designated CD4-kLC-pRcCMV (ATCC No. 75194; U.S. Pat. No. 6,187,748 B1 to Maddon and Beaudry, 2001). CD4-IgG2 has been shown to effectively neutralize a wide variety of HIV-1 isolates and can therefore potentially be used to inhibit HIV infection of a CD4+ cell, prevent a subject from becoming infected with HIV, and treat an HIV-infected subject so as to block the spread of HIV infection (U.S. Pat. No. 6,187,748 B1 to Maddon and Beaudry, 2001; Gauduin et al., 1996; Progenics, 2002).

The tetravalent CD4-IgG2 possesses increased serum half-life and increased avidity for HIV-1 as compared to sCD4 and the previous generation of mono- and divalent CD4-IgG chimeric proteins (U.S. Pat. No. 6,187,748 B1 to Maddon and Beaudry, 2001; Gauduin et al., 1996). However, as originally constituted in phosphate-buffered saline (PBS; 10 mM sodium phosphate, 140 mM sodium chloride, pH 7.0), the CD4-IgG2 molecule exhibited low stability upon storage which militated against the preparation of stable, highly concentrated liquid dosages that could be used for subcutaneous (SC) or intramuscular (IM) administration. These modes of administration enable simplified dosing options in HIV-infected individuals, but efforts to concentrate the drug in PBS resulted in the formation of aggregates and loss of activity. Thus, the previously prepared formulations of CD4-IgG2 were of relatively low concentration, typically <10 mg/ml. Moreover, the original formulation was incompatible with lyophilization of the drug for long-term storage and subsequent high recovery in its active form.

This disclosure describes the development of stable, liquid formulations containing higher concentrations of CD4-IgG2 than were prepared previously, i.e., at concentrations between about 25-162 mg/ml and which have been shown to be well tolerated and to have long half-lives (˜1 day) in rabbit studies. These formulations include highly concentrated versions, containing 100-162 mg/ml of CD4-IgG2, that have the potential for self-administration by HIV-infected individuals via SC and IM routes, as well as less concentrated formulations that are potentially valuable for intravenous (IV) administration.

### SUMMARY OF THE INVENTION

The present invention provides a pharmaceutical formulation optimized for lyophilization comprising a CD4-IgG2 chimeric heterotetramer, a histidine buffer and a maltose lyoprotectant, wherein the chimeric heterotetramer is present in the formulation at a concentration of between about 25-100 mg/ml and the formulation has a pH of between about 5.0-7.0.

This invention also provides a pharmaceutical formulation optimized for lyophilization comprising a CD4-IgG2 chimeric heterotetramer, a histidine buffer, a maltose lyoprotectant and an amino acid stabilizing agent, wherein the heterotetramer is present in the formulation at a concentration of between about 25-100 mg/ml and the formulation has a pH of between about 5.0-7.0.

This invention further provides a pharmaceutical formulation optimized for lyophilization comprising a CD4-IgG2 chimeric heterotetramer, a histidine buffer, a maltose lyoprotectant and a nonionic detergent, wherein the heterotetramer is present in the formulation at a concentration of between about 25-100 mg/ml and the formulation has a pH of between about 5.0-7.0.

This invention still further provides a pharmaceutical formulation optimized for lyophilization comprising a CD4-IgG2 chimeric heterotetramer, a histidine buffer, a maltose lyoprotectant and at least one osmolality adjusting agent, wherein the heterotetramer is present in the formulation at a concentration of between about 25-100 mg/ml and the formulation has a pH of between about 5.0-7.0.

This invention also provides a pharmaceutical formulation optimized for lyophilization comprising a CD4-IgG2 chimeric heterotetramer, a histidine buffer, a maltose lyoprotectant, an amino acid stabilizing agent and at least one osmolality adjusting agent, wherein the heterotetramer is present in the formulation at a concentration of between about 25-100 mg/ml, wherein the buffer is present in the formulation at a concentration of between about 3-15 mM, wherein the amino acid stabilizing agent is selected from the group consisting of alanine, glycine, proline and glycylglycine and is present in the formulation at a concentration of between about 10-30 mM, wherein the lyoprotectant is present in the formulation at a concentration of between about 1-5%, wherein the at least one osmolality adjusting agent is selected from the group consisting of maltose, trehalose and glycine and is present in the formulation at a concentration of between about 1-4%, and wherein this formulation has a pH of between about 5.0-7.0.

In addition, the present invention provides a reconstituted lyophilized pharmaceutical formulation comprising a CD4-IgG2 chimeric heterotetramer, a histidine buffer and a maltose stabilizer, wherein the chimeric heterotetramer is present in the formulation at a concentration of between about 100-162 mg/ml and the formulation has a pH of between about 5.0-7.0.

This invention also provides a reconstituted lyophilized pharmaceutical formulation comprising a CD4-IgG2 chimeric heterotetramer, a histidine buffer, a maltose stabilizer and an amino acid stabilizing agent, wherein the heterotetramer is present in the formulation at a concentration of between about 100-162 mg/ml and the formulation has a pH of between about 5.0-7.0.

This invention further provides a reconstituted lyophilized pharmaceutical formulation comprising a CD4-IgG2 chimeric heterotetramer, a histidine buffer, a maltose stabilizer and a nonionic detergent, wherein the heterotetramer is present in the formulation at a concentration of between about 100-162 mg/ml and the formulation has a pH of between about 5.0-7.0.

This invention still further provides a reconstituted lyophilized pharmaceutical formulation comprising a CD4-IgG2 chimeric heterotetramer, a histidine buffer, a maltose stabilizer and at least one osmolality adjusting agent, wherein the heterotetramer is present in the formulation at a concentration of between about 100-162 mg/ml and the formulation has a pH of between about 5.0-7.0.

This invention also provides a reconstituted lyophilized pharmaceutical formulation comprising a CD4-IgG2 chimeric heterotetramer, a histidine buffer, a maltose stabilizer, an amino acid stabilizing agent and at least one osmolality adjusting agent, wherein the heterotetramer is present in the formulation at a concentration of between about 100-162 mg/ml, wherein the buffer is present in the formulation at a concentration of between about 5-50 mM, wherein the maltose stabilizer is present in the formulation at a concentration of between about 1-10%, wherein the amino acid stabilizing agent is selected from the group consisting of alanine, glycine, proline and glycylglycine and is present in the formulation at a concentration of between about 25-150 mM, wherein the at least one osmolality adjusting agent is selected from the group consisting of maltose, trehalose and glycine and is present in the formulation at a concentration of between about 4-10%, and wherein this formulation has a pH of between about 5.0-7.0.

Additionally, this invention is directed to a method of inhibiting infection of a CD4+ cell by a human immunodeficiency virus, which method comprises contacting the human immunodeficiency virus with an amount of any of the above-described formulations effective to bind to such human immunodeficiency virus which is in the vicinity of the CD4+cell, so as to thereby inhibit infection of the CD4+ cell by the virus.

This invention is also directed to a method of preventing CD4+ cells of a subject from becoming infected with human immunodeficiency virus, which method comprises administering to the subject an amount of any of the above-described formulations effective to bind to human immunodeficiency virus present in the subject, so as to thereby prevent the subject's CD4+ cells from becoming infected with human immunodeficiency virus.

This invention is further directed to a method of treating a subject having CD4+ cells infected with human immunodeficiency virus which comprises administering to the subject an amount of any of the above-described formulations effective to bind to human immunodeficiency virus present in the subject, so as to thereby treat the subject having CD4+ cells infected with human immunodeficiency virus.

In addition, the present invention provides a method of making a pharmaceutical formulation comprising a CD4-IgG2 chimeric heterotetramer, which method comprises: (a) dissolving the heterotetramer in a solution comprising a histidine buffer at a pH of between about 5.0 and 7.0 and a maltose lyoprotectant at a concentration of between about 1-5% to produce a first formulation having a concentration of the heterotetramer of between about 25-100 mg/ml; (b) lyophilizing the first formulation to produce a lyophilized formulation; and (c) adding a diluent to the lyophilized formulation to produce a reconstituted pharmaceutical formulation containing a concentration of the heterotetramer of between about 100-162 mg/ml.

This invention also provides an article of manufacture comprising (a) a first packaging material containing a lyophilized pharmaceutical formulation according to any of the above-described embodiments, and (b) instructions for making a pharmaceutical formulation by adding a diluent to produce a reconstituted pharmaceutical formulation containing a CD4-IgG2 chimeric heterotetramer concentration of between about 100-162 mg/ml.

This invention further provides an article of manufacture comprising a packaging material containing therein a pharmaceutical formulation containing a CD4-IgG2 chimeric heterotetramer as described above and a label providing instructions for using this formulation in preventing infection of a subject by human immunodeficiency virus.

This invention still further provides an article of manufacture comprising a packaging material containing therein a pharmaceutical formulation containing a CD4-IgG2 chimeric heterotetramer as described above and a label providing instructions for using this formulation in treating subjects infected with human immunodeficiency virus.

This invention also provides a kit comprising a lyophilized pharmaceutical formulation corresponding to any of the formulations described above and a diluent for making a reconstituted pharmaceutical formulation by adding the diluent to the lyophilized formulation.

The instant invention further provides a kit comprising a pharmaceutical formulation according to any of the above-described embodiments and instructions for use.

### BRIEF DESCRIPTION OF THE FIGURES

FIG. 1. Schematic representation of CD4-IgG2 (PRO 542) protein. This chimeric, heterotetrameric protein comprises the constant domains C_{H}1, C_{H}2 and C_{H}3) of two IgG2 heavy chains fused to two gp120-binding domains of CD4 (V1 and V2), and the constant domains (C_{L}) of two kappa light chains fused to the V1 and V2 domains of CD4. Locations of N-linked glycosylation sites are indicated by black squares. The four chains in the tetrameric molecule are attached via disulfide (S—S) bonds.

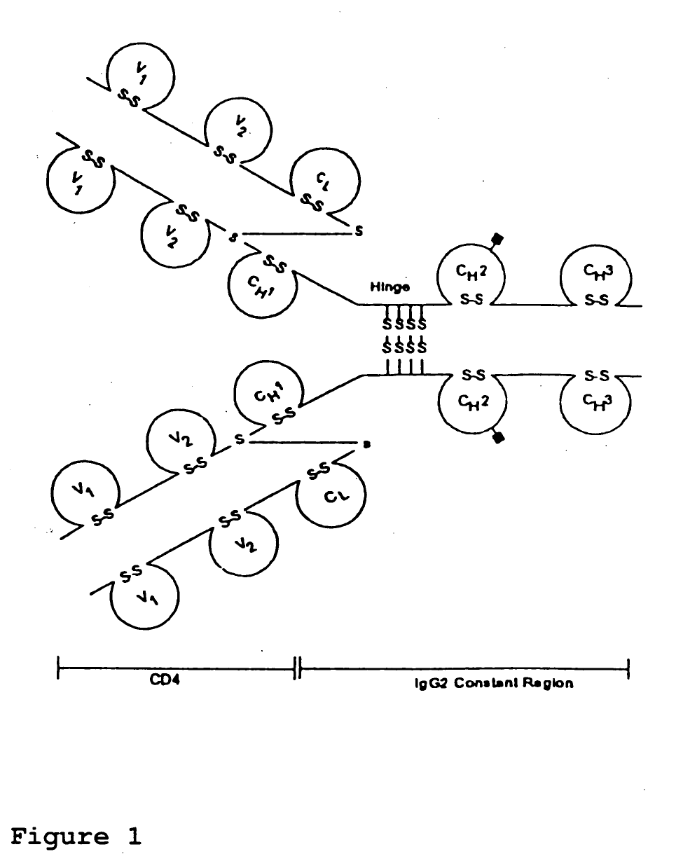

FIG. 2. HPLC-size exclusion chromatography of CD4-IgG2 formulations on a TSK G3000SW_{XL} column. In the depicted examples, a maltose lyoprotectant (6%) was present in the starting buffer prior to lyophilization of the CD4-IgG2 formulations. The chromatographs enable the quantitation and determination of purity (monomer vs. aggregate) of the CD4-IgG2 chimeric heterotetramer in the formulations before lyophilization (A) and after lyophilization (B).

FIG. 3. Changes in serum levels of CD4-IgG2 in inoculated rabbits over time. Formulations 0, 2 and 3 of CD4-IgG2 (see Table 3) were administered to rabbits and serum levels of the drug were assayed by sandwich ELISA over a period of five days. Terminal serum half-life was calculated by non-linear regression using WinNonLin (Pharsight Corporation, Mountain View, Calif.). The mean serum CD4-IgG2 levels are depicted for the cohort of animals that received the formulation by IV injection.

### DETAILED DESCRIPTION OF THE INVENTION

CD4-IgG2 is a novel HIV-1 attachment and entry inhibitor that has shown potent antiviral activity in Phase I/II clinical testing. However, previously constituted formulations of this therapeutic in PBS (phosphate-buffered saline; 10 mM sodium phosphate, pH 7.0, 140 mM sodium chloride) showed considerable instability over time, were incompatible with lyophilization and high recovery in its active form, and were at concentrations (<10 mg/ml CD4-IgG2) that were too low for SC or IM delivery. The invention described herein overcomes these limitations.

The present invention is directed to a pharmaceutical formulation optimized for lyophilization comprising a CD4-IgG2 chimeric heterotetramer, a histidine buffer and a maltose lyoprotectant, wherein the chimeric heterotetramer is present in this formulation at a concentration of between about 25-100 mg/ml and this formulation has a pH of between about 5.0-7.0. In one embodiment, the chimeric heterotetramer is present in this formulation at a concentration of between about 30-70 mg/ml. In a preferred embodiment, the heterotetramer is present in a concentration of about 50 mg/ml. In a further embodiment, the formulation has a pH of about 5.5-6.5. Preferably, the formulation has a pH of about 6.0. In another embodiment, the histidine buffer is present in this formulation at a concentration of between about 3-15 mM. In a further embodiment, the histidine buffer is present at a concentration of between about 5-10 mM. In a preferred embodiment, the histidine buffer is present at a concentration of about 6.7 mM.

In one embodiment of this invention, the lyoprotectant is present in the formulation at a concentration of between about 1-5%. As used herein, a percentage concentration shall mean grams per 100 milliliters (g/100 ml). In a further embodiment, the lyoprotectant is present at a concentration of between about 1-3%. In a preferred embodiment, the lyoprotectant is present at a concentration of about 2%.

Embodiments of the CD4-IgG2 formulations described herein may further comprise at least one additional anti-viral agent. In further embodiments, the at least one additional antiviral agent is selected from the group consisting of a non-nucleoside reverse transcriptase inhibitor (NNRTI), a nucleoside reverse transcriptase inhibitor, an HIV-1 protease inhibitor, a viral entry or fusion inhibitor, and combinations thereof.

The NNRTI compounds that may be used in the formulations of the present invention include but are not limited to efavirenz, UC-781, HBY 097, nevirapine (11-cyclopropyl-5,11,-dihydro-4-methyl-6H-dipyrido[3,2-b:2′3′-][1,4] diazepin-6-one), delavirdine (Rescriptor™; Pharmacia Upjohn) (piperazine, 1-[3-[(1-methyl-ethyl)amino]-2-pyridinyl]-4-[[5-[(methylsul-fonyl)amino]-1H-indol-2-yl]carbonyl]-, monomethanesulfonate), SJ-3366 (1-(3-cyclopenten-1-yl)methyl-6-(3,5-dimethylbenzoyl)-5-ethyl-2,4-pyrimidinedione), MKC-442 (6-benzyl-1-(ethoxy-methyl)-5-isopropyluracil), GW420867x (S-3 ethyl-6-fluro-4-isopropoxycarbonyl-3,4-dihydro-quinoxalin-2(1H)-one; Glaxo-SmithKline, Research Triangle Park, N.C.), HI-443 (N′-[2-(2-thiophene)ethyl]-N′-[2-(5-bromopyridyl)]-thiourea), and the like.

The nucleoside reverse transcriptase inhibitors that may be used in the formulations include but are not limited to abacavir (Ziagen™, GlaxoSmithKline) ((1S,cis)-4-[2-amino-6-(cyclopropylamino)-9H-purin-9-yl]-2-cyclopentene-1-methanol sulfate (salt)), lamivudine (Epivir™, GlaxoSmithKline) ((2R, cis)-4-amino-1-(2-hydroxymethyl-1,3-oxathiolan-5-yl)-(1H)-pyrimidin-2-one), zidovudine (Retrovir™; GlaxoSmithKline) (3′azido-3′-deoxythymidine), stavudine (Zerit; Bristol-Myers Squibb, New York, N.Y.) (2′,3′-didehydro-3′deoxythymidine), zacitabine (Hivid™; Hoffmann-La Roche, Nutley, N.J.) (4-amino-1-beta-D2′,3′-dideoxyribofuranosyl-2-(1H)-pyrimidone), didanosine, and the like.

The HIV-1 protease inhibitors that may be used in the formulations of the present invention include but are not limited to lopinavir (1S-[1R*,(R*),3R*,4R*]]-N-4-[[(2,6-dimethyphenoxy)acetyl] amino]-3-hydroxy-5-phenyl-1-(phenyl-methyl)pentyl] tetrahydro-alpha-(1-methylethyl)-2-oxol(2H)-pyrimidineacetamide), saquinavir (N-tert-butyl-decahydro-2-[2(R)-hydroxy-4-phenyl-3(S)-[[N-(2-quinolylcarbonyl)-L-asparaginyl]amino]butyl]-(4aS,8aS)-isoquinoline-(3S)-carboxamide), nelfinavir mesylate ([3S-[2(2S-,3S*),3a,4β,8aβ]]-N-(1,1-dimethyetyl)decahydro-2[2-hydroxy-3-[(3-hydroxy-2-methylbenzoyl)amino]-4-(phenylthio)-butyl]-3-isoquinolinecarboxamide mono-methane sulfonate), indinavir sulfate (([1(1S,2R),5(S))]-2,3,5-trideoxy-N-(2,3-dihydro-2-hydroxy-1H-inden-1-yl)-5-[2-[[(1,1-dimethylethyl)amino]carbonyl]-4-(3-pyridinylmethyl)-1-piperazinyl]-2-(phenylmethyl)-D-erythropentonamide sulfate (1:1) salt), amprenavir ((3S)-tetrahydro-3-furyl N-[(1S,2R)-3-(4-amino-N-isobutylbenzenesulfonamido)-1-benzyl-2-hydroxy-propyl]carbamate), ritonavir ((10-Hydroxy-2-methyl-5-(1-methylethyl)-1-[2-(1-methylethyl) -4-thiazolyl]-3,6-dioxo-8,11-bis (phenylmethyl)-2,4,7,12-tetraazatridecan-13-oic acid, 5-thiazolylmethyl ester, [5S-(5R*, 8R*, 10R*, 11R*)]), and the like.

The HIV entry or fusion inhibitors that may be used in the formulations of the present invention include but are not limited to an anti-CCR5 antibody (WO 03/072766 A1), T-20 (FUZEONO) and T-1249 (Trimeris, Inc., Durham, N.C.), UK-427,857 (Pfizer, Groton, Conn.), SCH-D (Schering-Plough, Kenilworth, N.J.), TAK-220 (Takeda Pharmaceuticals, Lincolnshire, Ill.), and AK602 (Ono Pharmaceuticals, Lawrenceville, N.J.).

The above-mentioned antiviral agents may be used in the methods of preventing or inhibiting HIV infection as described herein.

In another embodiment of this invention, the formulation is lyophilized. In a further embodiment, the lyophilized formulation is stable at ambient temperature for at least about twenty-four months.

As used herein, a “stable” formulation is one in which the structural integrity of CD4-IgG2 remains at least about 90% intact, substantially free of CD4-IgG2 aggregates and/or CD4-IgG2 degradation products, and in which at least about 90% of the antiviral activity of CD4-IgG2 is maintained. The structural integrity of CD4-IgG2 may be evaluated by various biophysical methods, including but not limited to, TSK size exclusion chromatography (TosoHaas, Montgomeryville, Pa.), sodium dodecyl sulfate-polyacrylamide gel electrophoresis (SDS-PAGE), by measuring the protein concentration as the net UV absorbance at a wavelength of 280 nm, and by visible inspection of liquid formulations for the presence of particulates against light and dark backgrounds. Methods for determining the antiviral activity of CD4-IgG2 include, for example, measuring the inhibition of HIV envelope glycoprotein-mediated cell fusion by the resonance energy transfer (RET) assay (Litwin et al., 1996) or by microscopically monitoring the inhibition of syncytium formation (Allaway et al., 1993), and measuring the affinity of the CD4-IgG2 product for gp120 by ELISA (Allaway et al., 1995).

As used herein, “substantially free” shall mean having 10% or less aggregation and/or degradation products, preferably 4% or less, more preferably 3% or less aggregation and/or degradation products. In a preferred embodiment, the formulation contains 2% or less aggregates and/or degradation products.

This invention also provides a pharmaceutical formulation optimized for lyophilization comprising a CD4-IgG2 chimeric heterotetramer, a histidine buffer, a maltose lyoprotectant and an amino acid stabilizing agent, wherein the heterotetramer is present in this formulation at a concentration of between about 25-100 mg/ml and this formulation has a pH of between about 5.0-7.0. In one embodiment, the chimeric heterotetramer is present in this formulation at a concentration of between about 30-70 mg/ml. In a preferred embodiment, the heterotetramer is present in a concentration of about 50 mg/ml. In a further embodiment, the formulation has a pH of about 5.5-6.5. Preferably, the formulation has a pH of about 6.0. In another embodiment, the histidine buffer is present in this formulation at a concentration of between about 3-15 mM. In a further embodiment, the histidine buffer is present at a concentration of between about 5-10 mM. In a preferred embodiment, the histidine buffer is present at a concentration of about 6.7 mM. In another embodiment of the instant invention, the lyoprotectant is present in the formulation at a concentration of between about 1-5%. In a further embodiment, the lyoprotectant is present at a concentration of between about 1-3%. In a preferred embodiment, the lyoprotectant is present at a concentration of about 2%.

Examples of amino acid stabilizing agents useful in the invention include but are not limited to alanine, glycine, proline and glycylglycine. In one embodiment of this invention, the amino acid stabilizing agent is present in this formulation at a concentration of between about 10-30 mM. In a preferred embodiment, the amino acid stabilizing agent is glycine and the glycine is present in the formulation at a concentration of about 17 mM.

In another embodiment of this invention, the formulation further comprises at least one additional anti-viral agent. In yet another embodiment, the at least one additional antiviral agent is selected from the group consisting of a non-nucleoside reverse transcriptase inhibitor (NNRTI), a nucleoside reverse transcriptase inhibitor, an HIV-1 protease inhibitor and a viral entry or fusion inhibitor. In a further embodiment, the formulation is lyophilized. In a still further embodiment, the lyophilized formulation is stable at ambient temperature for at least about twenty-four months.

This invention further provides a pharmaceutical formulation optimized for lyophilization comprising a CD4-IgG2 chimeric heterotetramer, a histidine buffer, a maltose lyoprotectant and a nonionic detergent, wherein the heterotetramer is present in this formulation at a concentration of between about 25-100 mg/ml and this formulation has a pH of between about 5.0-7.0. In one embodiment, the chimeric heterotetramer is present in this formulation at a concentration of between about 30-70 mg/ml. In a preferred embodiment, the heterotetramer is present in a concentration of about 50 mg/ml. In a further embodiment, the formulation has a pH of about 5.5-6.5. Preferably, the formulation has a pH of about 6.0.

In one embodiment of this invention, the formulation further comprises an amino acid stabilizing agent selected from the group consisting of alanine, glycine, proline and glycylglycine. In another embodiment, the amino acid stabilizing agent is present in the formulation at a concentration of between about 10-30 mM. In a preferred embodiment, the amino acid stabilizing agent is glycine and the glycine is present in said formulation at a concentration of about 17 mM. In a further embodiment, the nonionic detergent in this formulation comprises a polysorbate composition. In a preferred embodiment, the polysorbate composition is polyoxyethylenesorbitan monooleate, sold under the brand name of Tween 80®. In an additional embodiment, the nonionic detergent is present in the formulation at a concentration of between about 0.01-0.02%.

In yet another embodiment of this invention, the histidine buffer is present in this formulation at a concentration of between about 3-15 mM. In a further embodiment, the histidine buffer is present at a concentration of between about 5-10 mM. Preferably, the histidine buffer is present in this formulation at a concentration of about 6.7 mM. In one embodiment, the lyoprotectant is present in the formulation at a concentration of between about 1-5%. In another embodiment, the lyoprotectant is present at a concentration of between about 1-3%. In a preferred embodiment, the lyoprotectant is present at a concentration of about 2%.

In one embodiment of this invention, the formulation further comprises at least one additional anti-viral agent. In another embodiment, the at least one additional antiviral agent is selected from the group consisting of a non-nucleoside reverse transcriptase inhibitor (NNRTI), a nucleoside reverse transcriptase inhibitor, an HIV-1 protease inhibitor and a viral entry or fusion inhibitor. In a further embodiment, the formulation is lyophilized. In a still further embodiment, the lyophilized formulation is stable at ambient temperature for at least about twenty-four months.

This invention still further provides a pharmaceutical formulation optimized for lyophilization comprising a CD4-IgG2 chimeric heterotetramer, a histidine buffer, a maltose lyoprotectant and at least one osmolality adjusting agent, wherein the heterotetramer is present in this formulation at a concentration of between about 25-100 mg/ml and this formulation has a pH of between about 5.0-7.0. In one embodiment, the chimeric heterotetramer is present in this formulation at a concentration of between about 30-70 mg/ml. In a preferred embodiment, the heterotetramer is present in a concentration of about 50 mg/ml. In a further embodiment, the formulation has a pH of about 5.5-6.5. Preferably, the formulation has a pH of about 6.0.

This formulation may additionally comprise one or more amino acid stabilizing agents including alanine, glycine, proline and glycylglycine. In one embodiment, the amino acid stabilizing agent is present in this formulation at a concentration of between about 10-30 mM. In a preferred embodiment, the amino acid stabilizing agent is glycine and the glycine is present at a concentration of about 17 mM. In another embodiment of this formulation, the at least one osmolality adjusting agent is selected from the group consisting of maltose, trehalose and glycine. In a further embodiment, the at least one osmolality adjusting agent is present in this formulation in a concentration of between about 1-4%. In a preferred embodiment, the at least one osmolality adjusting agent is maltose and the osmolality adjusting agent is present in the formulation at a concentration of between about 2-3%. In a still further embodiment, the at least one osmolality adjusting agent is present in the formulation in a concentration to provide an osmolality of about 70-110 mOsm/kg.

In an additional embodiment of this invention, the histidine buffer is present in this formulation at a concentration of between about 3-15 mM. In another embodiment, the histidine buffer is present at a concentration of between about 5-10 mM. Preferably, the histidine buffer is present in this formulation at a concentration of about 6.7 mM. In a further embodiment, the lyoprotectant is present in the formulation at a concentration of between about 1-5%. In a still further embodiment, the lyoprotectant is present at a concentration of between about 1-3%. In a preferred embodiment, the lyoprotectant is present at a concentration of about 2%.

In another embodiment of this invention, the formulation further comprises a nonionic detergent. In a further embodiment, the nonionic detergent comprises a polysorbate composition. In a preferred embodiment, the polysorbate composition is polyoxyethylenesorbitan monooleate, sold under the brand name of Tween 80®. In a still further embodiment, the nonionic detergent is present in this formulation at a concentration of between about 0.01-0.02%.

In another embodiment, the formulation further comprises at least one additional anti-viral agent. In a further embodiment, the at least one additional antiviral agent is selected from the group consisting of a non-nucleoside reverse transcriptase inhibitor (NNRTI), a nucleoside reverse transcriptase inhibitor, an HIV-1 protease inhibitor and a viral entry or fusion inhibitor. In a still further embodiment, the formulation is lyophilized. In an additional embodiment, the lyophilized formulation is stable at ambient temperature for at least about twenty-four months.

This invention also provides a pharmaceutical formulation optimized for lyophilization comprising a CD4-IgG2 chimeric-heterotetramer, a histidine buffer, a maltose lyoprotectant, an amino acid stabilizing agent and at least one osmolality adjusting agent, wherein the heterotetramer is present in this formulation at a concentration of between about 25-100 mg/ml, wherein the buffer is present in this formulation at a concentration of between about 3-15 mM, wherein the amino acid stabilizing agent is selected from the group consisting of alanine, glycine, proline and glycylglycine and is present in this formulation at a concentration of between about 10-30 mM, wherein the lyoprotectant is present in the formulation at a concentration of between about 1-5%, wherein the at least one osmolality adjusting agent is selected from the group consisting of maltose, trehalose and glycine and is present in the formulation at a concentration of between about 1-4%, and wherein this formulation has a pH of between about 5.0-7.0.

In one embodiment of this invention, the chimeric heterotetramer is present in this formulation at a concentration of between about 30-70 mg/ml. In a preferred embodiment, the heterotetramer is present in a concentration of about 50 mg/ml. In another embodiment, the formulation further comprises a nonionic detergent. In a further embodiment, the nonionic detergent comprises a polysorbate composition. In an additional embodiment, the at least one osmolality adjusting agent is present in the formulation at a concentration to provide an osmolality of about 70-110 mOsm/kg.

In another embodiment, the formulation further comprises at least one additional anti-viral agent. In a further embodiment, the at least one additional antiviral agent is selected from the group consisting of a non-nucleoside reverse transcriptase inhibitor (NNRTI), a nucleoside reverse transcriptase inhibitor, an HIV-1 protease inhibitor and a viral entry or fusion inhibitor. In a still further embodiment, the formulation is lyophilized. In yet another embodiment, the lyophilized formulation is stable at ambient temperature for at least about twenty-four months.

In addition, the present invention provides a reconstituted lyophilized pharmaceutical formulation comprising a CD4-IgG2 chimeric heterotetramer, a histidine buffer and a maltose stabilizer, wherein the chimeric heterotetramer is present in the formulation at a concentration of between about 100-162 mg/ml and the formulation has a pH of between about 5.0-7.0. As used herein, a “reconstituted lyophilized pharmaceutical formulation” shall mean a pharmaceutical formulation resulting from the dissolution, i.e. reconstitution, of a lyophilized sample in a suitable solution or diluent.

In one embodiment, the chimeric heterotetramer is present in this formulation at a concentration of between about 130-155 mg/ml. In a preferred embodiment, the heterotetramer is present in a concentration of about 150 mg/ml. In a further embodiment, the formulation has a pH of about 5.5-6.5. Preferably, the formulation has a pH of about 6.0. In another embodiment, the histidine buffer is present in this formulation at a concentration of between about 5-50 mM. In a further embodiment, the histidine buffer is present at a concentration of between about 10-30 mM. In a preferred embodiment, the histidine buffer is present at a concentration of about 20 mM. In an additional embodiment, the maltose stabilizer is present in the instant formulation at a concentration of between about 1-10%. In another embodiment, the maltose stabilizer is present at a concentration of between about 4-8%. In a preferred embodiment, the maltose stabilizer is present at a concentration of about 6%.

In another embodiment of this invention, the formulation further comprises at least one additional anti-viral agent. In a further embodiment, the at least one additional antiviral agent is selected from the group consisting of a non-nucleoside reverse transcriptase inhibitor (NNRTI), a nucleoside reverse transcriptase inhibitor, an HIV-1 protease inhibitor and a viral entry or fusion inhibitor. In a still further embodiment, the reconstituted formulation is stable at a temperature of 25° C. or less for at least about two months. In yet another embodiment, the formulation is stable at a temperature of 4° C. or less for at least about six months. In an additional embodiment, the formulation is stable at a temperature of −90° C. for at least about six months.

In one embodiment, the reconstituted formulation may be combined with a sealable container containing the formulation. In another embodiment, the formulation may be combined with a vial or ampoule containing the formulation, this vial or ampoule having a septum. In a further embodiment, the formulation may be combined with a sealable bottle containing the formulation. In a still further embodiment, the formulation may be combined with a syringe containing the formulation. In an additional embodiment, the formulation may be combined with an infusion bag containing this formulation. In another embodiment, the formulation is suitable for parenteral administration. In yet another embodiment, the parenteral administration is performed intravenously, subcutaneously or intramuscularly.

In an embodiment of the present invention, the reconstituted formulation is substantially free of CD4-IgG2 chimeric heterotetramer aggregates and degradation products. In another embodiment, the CD4-IgG2 chimeric heterotetramer is in at least 96% monomeric form. In a further embodiment, the CD4-IgG2 chimeric heterotetramer is in at least 97% monomeric form. In a still further embodiment, the CD4-IgG2 chimeric heterotetramer is in at least 98% monomeric form. In an additional embodiment, the CD4-IgG2 chimeric heterotetramer is in at least 99% monomeric form.

This invention also provides a reconstituted lyophilized pharmaceutical formulation comprising a CD4-IgG2 chimeric heterotetramer, a histidine buffer, a maltose stabilizer and an amino acid stabilizing agent, wherein the heterotetramer is present in this formulation at a concentration of between about 100-162 mg/ml and the formulation has a pH of between about 5.0-7.0. In one embodiment, the chimeric heterotetramer is present in this formulation at a concentration of between about 130-155 mg/ml. In a preferred embodiment, the heterotetramer is present at a concentration of about 150 mg/ml. In a further embodiment, the formulation has a pH of about 5.5-6.5. Preferably, the formulation has a pH of about 6.0. In an additional embodiment, the histidine buffer is present in this reconstituted formulation at a concentration of between about 5-50 mM. In another embodiment, the histidine buffer is present at a concentration of between about 10-30 mM. In a preferred embodiment, the histidine buffer is present at a concentration of about 20 mM.

In another embodiment of this invention, the maltose stabilizer is present in the reconstituted formulation at a concentration of between about 1-10%. In a further embodiment, the maltose stabilizer is present at a concentration of between about 4-8%. In a preferred embodiment, the maltose stabilizer is present at a concentration of about 6%.

This reconstituted formulation may additionally comprise one or more amino acid stabilizing agents selected from the group consisting of alanine, glycine, proline and glycylglycine. In one embodiment, the amino acid stabilizing agent is present in this formulation at a concentration of between about 25-150 mm. In a preferred embodiment, the amino acid stabilizing agent is glycine and the glycine is present at a concentration of about 50 mM. In another embodiment of this invention, the formulation further comprises at least one additional anti-viral agent. In yet another embodiment, the at least one additional antiviral agent is selected from the group consisting of a non-nucleoside reverse transcriptase inhibitor (NNRTI), a nucleoside reverse transcriptase inhibitor, an HIV-1 protease inhibitor and a viral entry or fusion inhibitor.

This invention further provides a reconstituted lyophilized pharmaceutical formulation comprising a CD4-IgG2 chimeric heterotetramer, a histidine buffer, a maltose stabilizer and a nonionic detergent, wherein the heterotetramer is present in this formulation at a concentration of between about 100-162 mg/ml and the formulation has a pH of between about 5.0-7.0.

In one embodiment, the chimeric heterotetramer is present in this formulation at a concentration of between about 130-155 mg/ml. Preferably, the chimeric heterotetramer is present at a concentration of about 150 mg/ml. In another embodiment, the formulation has a pH of about 5.5-6.5. In a preferred embodiment, the reconstituted formulation has a pH of about 6.0.

This formulation may additionally comprise one or more amino acid stabilizing agents selected from the group consisting of alanine, glycine, proline and glycylglycine. In one embodiment, the amino acid stabilizing agent is present in this formulation at a concentration of between about 25-150 mM. In a preferred embodiment, the amino acid stabilizing agent is glycine and the glycine is present at a concentration of about 50 mM. In another embodiment, the nonionic detergent comprises a polysorbate composition. In a preferred embodiment, the polysorbate composition is polyoxyethylenesorbitan monooleate, sold under the brand name of Tween 80®. In a further embodiment, the nonionic detergent is present in this formulation at a concentration of between about 0.02-0.05%. Preferably, the nonionic detergent is present at a concentration of about 0.05%.

In another embodiment of the instant invention, the histidine buffer is present in this reconstituted formulation at a concentration of between about 5-50 mM. In a further embodiment, the histidine buffer is present at a concentration of between about 10-30 mM. In a preferred embodiment, the histidine buffer is present at a concentration of about 20 mM. In another embodiment, the maltose stabilizer is present in the formulation at a concentration of between about 1-10%. In a further embodiment, the maltose stabilizer is present at a concentration of between about 4-8%. In a preferred embodiment, the maltose stabilizer is present at a concentration of about 6%.

In another embodiment, the formulation further comprises at least one additional anti-viral agent. In an additional embodiment, the at least one additional antiviral agent is selected from the group consisting of a non-nucleoside reverse transcriptase inhibitor (NNRTI), a nucleoside reverse transcriptase inhibitor, an HIV-1 protease inhibitor and a viral entry or fusion inhibitor.

This invention further provides a reconstituted lyophilized pharmaceutical formulation comprising a CD4-IgG2 chimeric heterotetramer, a histidine buffer, a maltose stabilizer and at least one osmolality adjusting agent, wherein the heterotetramer is present in the formulation at a concentration of between about 100-162 mg/ml and this formulation has a pH of between about 5.0-7.0. In one embodiment, the chimeric heterotetramer is present in this formulation at a concentration of between about 130-155 mg/ml. Preferably, the heterotetramer is present at a concentration of about 150 mg/ml. In a further embodiment, the formulation has a pH of about 5.5-6.5. In a preferred embodiment, the formulation has a pH of about 6.0.

In another embodiment, this reconstituted formulation further comprises one or more amino acid stabilizing agents selected from the group consisting of alanine, glycine, proline and glycylglycine. In a further embodiment, the amino acid stabilizing agent is present in this formulation at a concentration of between about 25-150 mM. In a preferred embodiment, the amino acid stabilizing agent is glycine and the glycine is present at a concentration of about 50 mM. In an additional embodiment, the histidine buffer is present in this reconstituted formulation at a concentration of between about 5-50 mM. In another embodiment, the histidine buffer is present at a concentration of between about 10-30 mM. In a preferred embodiment, the histidine buffer is present at a concentration of about 20 mM. In a further embodiment, the maltose stabilizer is present in the formulation at a concentration of between about 1-10%. In a further embodiment, the maltose stabilizer is present at a concentration of between about 4-8%. In a preferred embodiment, the maltose stabilizer is present at a concentration of about 6%.

In one embodiment of the present formulation, the at least one osmolality adjusting agent is selected from the group consisting of maltose, trehalose and glycine. In another embodiment, the at least one osmolality adjusting agent is present in the formulation at a concentration of between about 4-10%. In a preferred embodiment, the at least one osmolality adjusting agent is maltose and the osmolality adjusting agent is present at a concentration of between about 6-7%. In a further embodiment, the at least one osmolality adjusting agent is present in the formulation in a concentration to provide an osmolality of about 216-320 mOsm/kg. In a still further embodiment, the at least one osmolality adjusting agent is present at a concentration to provide an osmolality of about 220-300 mOsm/kg. In yet another embodiment, the at least one osmolality adjusting agent is present at a concentration to provide an osmolality of about 280 mOsm/kg.

In an additional embodiment, this reconstituted formulation further comprises a nonionic detergent. In another embodiment, the nonionic detergent comprises a polysorbate composition. In a preferred embodiment, the polysorbate composition is polyoxyethylenesorbitan monooleate, sold under the brand name of Tween 80®. In a further embodiment, the nonionic detergent is present in this formulation at a concentration of between about 0.02-0.05%. Preferably, the nonionic detergent is present at a concentration of about 0.05%.

In one embodiment of this invention, the formulation further comprises at least one additional anti-viral agent. In another embodiment, the at least one additional antiviral agent is selected from the group consisting of a non-nucleoside reverse transcriptase inhibitor (NNRTI), a nucleoside reverse transcriptase inhibitor, an HIV-1 protease inhibitor and a viral entry or fusion inhibitor.

This invention still further provides a reconstituted lyophilized pharmaceutical formulation comprising a CD4-IgG2 chimeric heterotetramer, a histidine buffer, a maltose stabilizer, an amino acid stabilizing agent and at least one osmolality adjusting agent, wherein the heterotetramer is present in this formulation at a concentration of between about 100-162 mg/ml, wherein the buffer is present in the formulation at a concentration of between about 5-50 mM, wherein the maltose stabilizer is present in the formulation at a concentration of between about 1-10%, wherein the amino acid stabilizing agent is selected from the group consisting of alanine, glycine, proline and glycylglycine and is present in the formulation at a concentration of between about 25-150 mM, wherein the at least one osmolality adjusting agent is selected from the group consisting of maltose, trehalose and glycine and is present in the formulation at a concentration of between about 4-10%, and wherein this formulation has a pH of between about 5.0-7.0. In one embodiment of this reconstituted formulation, the chimeric heterotetramer is present in the formulation at a concentration of between about 130-155 mg/ml. In another embodiment, the chimeric heterotetramer is present in the formulation at a concentration of about 150 mg/ml.

In a further embodiment, the formulation further comprises a nonionic detergent. In a still further embodiment, the nonionic detergent comprises a polysorbate composition. In an additional embodiment, the at least one osmolality adjusting agent is present in the formulation at a concentration to provide an osmolality of about 216-320 mOsm/kg. In a further embodiment, the at least one osmolality adjusting agent is present at a concentration to provide an osmolality of about 220-300 mOsm/kg. In a still further embodiment, the at least one osmolality adjusting agent is present at a concentration to provide an osmolality of about 280 mOsm/kg.

In another embodiment of this invention, the formulation further comprises at least one additional anti-viral agent. In yet another embodiment, the at least one additional antiviral agent is selected from the group consisting of a non-nucleoside reverse transcriptase inhibitor (NNRTI), a nucleoside reverse transcriptase inhibitor, an HIV-1 protease inhibitor and a viral entry or fusion inhibitor.

Additionally, this invention is directed to a method of inhibiting infection of a CD4+ cell by a human immunodeficiency virus, which method comprises contacting the human immunodeficiency virus with an amount of any of the above-described formulations effective to bind to such human immunodeficiency virus which is in the vicinity of the CD4+ cell, so as to thereby inhibit infection of the CD4+ cell by the virus.

This invention is also directed to a method of preventing CD4+ cells of a subject from becoming infected with human immunodeficiency virus, which method comprises administering to the subject an amount of any of the above-described formulations effective to bind to human immunodeficiency virus present in the subject, so as to thereby prevent the subject's CD4+ cells from becoming infected with human immunodeficiency virus.

This invention is further directed to a method of treating a subject having CD4+ cells infected with human immunodeficiency virus which comprises administering to the subject an amount of any of the above-described formulations effective to bind to human immunodeficiency virus present in the subject, so as to thereby treat the subject having CD4+ cells infected with human immunodeficiency virus.

In addition, the present invention provides a method of making a pharmaceutical formulation comprising a CD4-IgG2 chimeric heterotetramer, which method comprises: (a) dissolving the heterotetramer in a solution comprising a histidine buffer at a pH of between about 5.0 and 7.0 and a maltose lyoprotectant at a concentration of between about 1-5% to produce a first formulation having a concentration of the heterotetramer of between about 25-100 mg/ml; (b) lyophilizing the first formulation to produce a lyophilized formulation; and (c) adding a diluent to the lyophilized formulation to produce a reconstituted pharmaceutical formulation containing a concentration of the heterotetramer of between about 100-162 mg/ml. In one embodiment, the chimeric heterotetramer is present in the formulation at a concentration of about 130-155 mg/ml. In a preferred embodiment, the concentration of the chimeric heterotetramer is about 150 mg/ml.

In another embodiment of the instant method, the maltose lyoprotectant is present in the reconstituted pharmaceutical formulation at a concentration of between about 1-10%. In a further embodiment, the maltose lyoprotectant is present in the formulation at a concentration of between about 4-8%. Preferably, the maltose lyoprotectant is present in the reconstituted pharmaceutical formulation at a concentration of about 6%. In a still further embodiment, the diluent is water-for-injection or physiological saline.

Another embodiment of the present method further comprises adding to the first formulation at least one of an amino acid stabilizing agent, a nonionic detergent, at least one osmolality adjusting agent, and at least one additional anti-viral agent, including combinations thereof. In a further embodiment, the amino acid stabilizing agent is selected from the group consisting of alanine, glycine, proline and glycylglycine and is admixed in the first formulation at a concentration of between about 10-30 mM. In another embodiment, the nonionic detergent comprises a polysorbate composition and is admixed in the first formulation at a concentration of between about 0.01-0.02%. In a further embodiment, the at least one osmolality adjusting agent is selected from the group consisting of maltose, trehalose and glycine and is admixed in the first formulation at a concentration of between about 1-5%. In a still further embodiment, the at least one osmolality adjusting agent is present in this formulation at a concentration to provide an osmolality of about 70-110 mOsm/kg. In yet another embodiment, the at least one additional antiviral agent is selected from the group consisting of a non-nucleoside reverse transcriptase inhibitor (NNRTI), a nucleoside reverse transcriptase inhibitor, an HIV-1 protease inhibitor and a viral entry or fusion inhibitor, and is admixed in the first formulation.

In a further embodiment of this method, the amino acid stabilizing agent is selected from the group consisting of alanine, glycine, proline and glycylglycine and is present in the reconstituted pharmaceutical formulation at a concentration of between about 25-150 mM. In a still further embodiment, the amino acid stabilizing agent is glycine and the glycine is present in the reconstituted pharmaceutical formulation at a concentration of about 50 mM. In an additional embodiment, the nonionic detergent is present in the reconstituted pharmaceutical formulation at a concentration of between about 0.02-0.05%. Preferably, the nonionic detergent is present at a concentration of about 0.05%. In another embodiment, the at least one osmolality adjusting agent is selected from the group consisting of maltose, trehalose and glycine and is present in the reconstituted pharmaceutical formulation at a concentration of between about 4-10%.

In yet another embodiment, the at least one osmolality adjusting agent is maltose and is present in the reconstituted pharmaceutical formulation at a concentration of between about 6-7%. In a further embodiment, the at least one osmolality adjusting agent is present in said reconstituted pharmaceutical formulation at a concentration to provide an osmolality of about 216-320 mOsm/kg. In a still further embodiment, the at least one osmolality adjusting agent is present at a concentration to provide an osmolality of about 220-300 mOsm/kg. In a preferred embodiment, the at least one osmolality adjusting agent is present at a concentration to provide an osmolality of about 280 mOsm/kg. In another embodiment, the at least one additional antiviral agent is selected from the group consisting of a non-nucleoside reverse transcriptase inhibitor (NNRTI), a nucleoside reverse transcriptase inhibitor, an HIV-1 protease inhibitor and a viral entry or fusion inhibitor, and is present in the reconstituted pharmaceutical formulation.

This invention is also directed to an article of manufacture comprising: (a) a first packaging material containing a lyophilized pharmaceutical formulation according to any of the above-described embodiments, and (b) instructions for making a pharmaceutical formulation by adding a diluent to produce a reconstituted pharmaceutical formulation containing a CD4-IgG2 chimeric heterotetramer concentration of between about 100-162 mg/ml. One embodiment of this article of manufacture further comprises a second packaging material containing a diluent. In another embodiment, the diluent is water-for-injection or physiological saline.

This invention is further directed to an article of manufacture comprising a packaging material containing therein a pharmaceutical formulation containing a CD4-IgG2 chimeric heterotetramer as described above and a label providing instructions for using this formulation in preventing infection of a subject by human immunodeficiency virus.

This invention still further provides an article of manufacture comprising a packaging material containing therein a pharmaceutical formulation containing a CD4-IgG2 chimeric heterotetramer as described above and a label providing instructions for using this formulation in treating subjects infected with human immunodeficiency virus.

In addition, this invention provides a kit comprising a lyophilized pharmaceutical formulation as described above and a diluent for making a reconstituted pharmaceutical formulation by adding the diluent to the lyophilized formulation. In one embodiment, the diluent is water-for-injection, physiological saline, or other diluent suitable for parenteral administration. Another embodiment further includes instructions for use.

The present invention also provides a kit comprising a pharmaceutical formulation according to any of the above-described embodiments and instructions for use.

Experimental Details

The following Experimental Details are set forth to aid in an understanding of the invention, and are not intended, and should not be construed, to limit in any way the invention set forth in the claims which follow thereafter.

Materials and Methods

Assessment of Molecular Structural Stability

The following techniques and procedures were used to profile the structural stability of CD4-IgG2 against changes in certain variables, namely pH, temperature, ionic strength, shear stress and freeze/thaw cycling.

Measurement of Intrinsic Fluorescence

Measurement of intrinsic fluorescence (IF) is based on detecting the fluorescence of the amino acid tryptophan to monitor protein structure. The fluorescence characteristics of tryptophan depend on its location within the protein; as the protein denatures, tryptophan residues normally buried within the protein are relocated into a more polar environment which shifts the emission spectra and changes the emission ratio. Monitoring of the emission ratio can yield information about the unfolding/structural response of the protein to external stimuli: the higher the ratio, the more unfolded the molecule.

A guanidine titration was performed to determine which combination of emission wavelengths would produce the maximum sensitivity (and therefore was most appropriate for use in future IF experiments). Samples were prepared at 0.2 mg/ml CD4-IgG2 in the original PBS formulation, and with 1 M, 2 M, 3 M, 4 M, 5 M and 6 M guanidine. Using an excitation wavelength of 295 nm, emission was monitored at 318 nm, 338 nm and 358 nm. A plot of emission intensity ratios vs. the molarity of guanidine in the sample was generated.

To determine the excitation and emission wavelengths for IF, 0.2 mg/ml CD4-IgG2 in PBS was prepared. An absorbance scan, using a UV spectrophotometer, indicated that maximum absorbance was at 282 nm. Thus, the excitation wavelength was set at 282 nm and an emission scan was determined using fluorescence spectroscopy. The maximum emission was found to be at 338 nm. This allowed the setting of parameters for monitoring changes around the tryptophan environment (for IF; see Table 1). Extrinsic fluorescence and RALS parameters, which are predetermined regardless of the characteristics of the molecule being assayed, Pymal and RET assay parameters are also included in Table 1.

TABLE 1
Excitation and emission wavelengths for fluorimeter tests

| | Excitation | Emission |
|---|---|---|
| Measurement | wavelength (nm) | wavelengths (nm) |
| Intrinsic Fluorescence (IF) | 295 | 318, 338, and 358* |
| Extrinsic Fluorescence (EF) | 380 | 490 and 520 |
| Right Angle Light | 320 | 320 |
| Scattering (RALS) | | |
| Pymal | 342 | 374 and 394 |
| Resonance Energy Transfer | 450 | 590 |
| (RET) | | |

*Wavelengths chosen based on determined maximum emission wavelength

Measurement of Extrinsic Fluorescence

Measurement of extrinsic fluorescence is based on the chromophore, 8-anilino-naphthalene sulphonic acid (ANS), whose fluorescence is enhanced when exposure to water is reduced. ANS binds to proteins by a combination of electrostatic and hydrophobic modes. As the protein unfolds, it exposes more hydrophobic pockets allowing more ANS to bind. The resulting enhanced fluorescence can be measured by monitoring changes in the emission ratio 520/490 nm. The lower this ratio, the more hydrophobic the molecule or the greater the unfolding which reveals hydrophobic pockets.

For EF measurements, ANS was added to the sample as an external probe. A titration was performed in which 1 mM ANS (in methanol) was added in increments to 400 μl of 0.2 mg/ml CD4-IgG2 in PBS. This was done to determine the ANS concentration at which the emission ratio plateaus. Fluorescence intensities at each ANS concentration were measured using a 380 nm excitation wavelength and 490 and 520 nm emission wavelengths.

Measurement of Right Angle Light Scattering (RALS)

The intensity of RALS is a measure of the aggregation present in a sample. The RALS measurement increases on a scale of 0-10 (instrument range) with increasing turbidity of a solution. RALS is often measured during a temperature ramp cycle in which temperature is increased usually from 20 to −80° C. With increasing temperature the sample tends to denature and then aggregate. The initial absolute intensity value and the transition temperature (temperature at which slope first changes) are indicative of the sample's initial aggregation status. The RALS value is dependent on the voltage setting of the instrument. Only samples analyzed at the same concentration and voltage and on the same instrument can be compared.

Temperature Profiling

Samples were placed in a cuvette and heated in a circulating water bath programmed to increase by 2° C./minute from 20° C. to 90° C. RALS, IF and EF readings were recorded at 0.5 or 1 minute intervals.

Assessing Effects of Freeze-Thaw Cycles

A 0.2 mg/ml solution of CD4-IgG2 was prepared from original stock material (5.4 mg/ml, never thawed) in PBS. The solution was divided into 2-ml aliquots which were subjected to successive freeze/thaw cycles, up to 5 times. Each freeze was performed at -80° C. for at least 50 minutes, and each thaw was performed at room temperature or 4° C. After each freeze-thaw cycle, the samples were analyzed by SEC-HPLC (size exclusion chromatography-high performance liquid chromatography), RALS, IF and EF.

Evaluation of Effects of Ionic Strength

A solution of CD4-IgG2 (5.4 mg/ml) in PBS was dialyzed into ix Base Buffer (BB; 2.0 mM of each of glycine, citric acid, Hepes, MES, Tris base, pH 7) containing 0, 10, 100, and 500 mM NaCl. A control sample was left in the original PBS formulation. The CD4-IGG2 concentration for these samples was then determined by UV spectrophotometry. Samples were diluted into their respective buffers to 0.2 mg/ml, aliquoted, and placed at 50° C. “T₀” samples were frozen at −80° C. immediately. At day 3, day 7, and day 14, additional samples were removed and placed at −80° C. until analysis.

Evaluation of Effects of pH

A solution of CD4-IgG2 (5.4 mg/mi) was diluted to 0.2 mg/ml into seven different preparations of 1×BB. The BB preparations contained ˜25 mM NaCl and ranged in pH from 4 to 8. A control sample was diluted into PBS. Samples were aliquoted and placed at 50° C. “T₀” samples were frozen -at −80° C. immediately. At day 3, day 7, and day 14, additional samples were removed and placed at −80° C. until analysis.

Evaluation of Shear Stress

Selected samples from the ionic strength evaluation and pH evaluation studies were evaluated for recovery, purity, and RALS properties after exposure to shear stress. Shear stress was simulated by placing each sample in a conical glass vial containing a triangular magnetic stir bar. Vials were placed on a stir plate set at approximately 300 rpm and allowed to stir without cavitation for 0.5-24 hours. Solution clarity was observed at various time points throughout the experiment. At the end of the experiment, samples were evaluated using RALS, then centrifuged and further analyzed by HPLC-SEC and UV spectrophotometry (for the pH evaluation study).

pH Titration

pH titration involved exposing the sample to extreme pH's (from pH 6 or 7, to pH 11, to 3, then back to 6 or 7) by adding small volumes of NaOH or HCl. Base was added, then immediately after the appropriate pH was reached additional acid was added to bring the sample to the next target pH, then base was added again to increase the pH. At the end of the experiment, samples were evaluated using RALS, then centrifuged and analyzed by HPLC-SEC.

Effect of 4° C. Incubation on CD4-IgG2 High Molecular Weight/Monomer Profile

An experiment was performed to evaluate the effect of incubation at 4° C. on the high molecular weight/monomer profile. CD4-IgG2 stock solution was diluted to 0.2 mg/ml in PBS. 1-ml aliquots were placed at −80° C. (controls) or 50° C. (temperature-stressed) immediately. After 7 days, the 50° C. samples were placed at −80° C. overnight. Over the following 2 days, samples were thawed at various times and allowed to incubate at 4-5° C. until HPLC-SEC analysis. Total incubation times before injection were approximately 1.5, 12, 28 and 38 h.

Determination of the Presence of Free Sulfhydryl Groups

A sulfhydryl probe, pyrene maleimide (Pymal), was used to detect the possible presence of free sulfhydryl groups in CD4-IgG2. Pymal in the absence of covalent reaction with free sulfhydryl groups is virtually fluorescence-free. However, following reaction with a sulfhydryl group, a characteristic 2-peak emission spectrum is seen with emission maxima at 374 nm and 394 nm.

CD4-IgG2 solutions (0.2 mg/ml) in PBS ±1% SDS and in 6M guanidine and corresponding blank solutions were prepared. A 50-fold molar excess of Pymal was added to each solution and fluorescence measurements, using a 342 nm excitation wavelength and a 370 to 410 nm emission scan, were made every 15 minutes for up to 105 minutes. Emissions at 374 nm and 394 nm were recorded. For each time point, the blank was subtracted from the corresponding protein-added sample to obtain an emission value that was used for an emission versus time plot.

Stability Study Iterations

The following screening protocol was used for stability study iterations:

1. A solution of CD4-IgG2 (5.1-5.4 mg/ml) in PBS was dialyzed into each buffer test condition or base buffer condition to which excipients were added later;

2. A control sample was left in PBS;

3. The concentration of the CD4-IgG2 dialysate was measured by UV spectrophotometry;

4. Dialysates were diluted (if necessary) into their respective buffers to 1 or 4.8 mg/ml and then aliquoted into 1.5 ml polypropylene microcentrifuge tubes;

5. “T₀” samples were frozen at −80° C. immediately;

6. The remainder of the samples were incubated at 50° C. for 7 (“T₇”) and 14 days (“T₁₄”) and then placed at −80° C. until analysis;

7. Samples at T₀, T₇ and T₁₄ (if necessary) were analyzed by measuring the UV absorbance at 280 nm, RALS, and by HPLC-SEC;

8. Samples were also analyzed by A₂₈₀ measurement, HPLC-SEC and RALS after shear stress.

Stabilizer samples were prepared for pH titration by dialyzing a CD4-IgG2 stock solution (5.4 mg/ml) in PBS into 20 mM histidine, pH 6. The samples were then diluted 10- or 12-fold to 0.45 mg/ml into the appropriate test buffer.

For buffer evaluation in iteration 1, CD4-IgG2 was dialyzed into 1) 20 mM citrate, pH 5.5; 2) 20 mM citrate, pH 6.0; 3) 20 mM histidine, pH 5.5; 4) 20 mM histidine, pH 6.0; and 5) 20 mM histidine, pH 6.5. CD4-IgG2 dialysates were diluted to approximately 1 mg/ml in each of their corresponding buffers.

For the evaluation of stabilizers in iteration 2, CD4-IgG2 was dialyzed into 10 different buffers each consisting of 20 mM histidine plus 20 mM of alanine, arginine, creatinine, glycine, proline, isoleucine, leucine, lysine, glycylglycine or no amino acid as a control. CD4-IgG2 dialysates were diluted to approximately 1 mg/ml in each of their corresponding buffers.

For the evaluation of stabilizers/bulking agents/lyoprotectants in iteration 3, CD4-IgG2 was dialyzed into 8 different buffers each consisting of 20 mM histidine. plus 3% of mannitol, lactose, sucrose, maltose, trehalose, sorbitol, fructose, or no carbohydrate as a control. CD4-IgG2 dialysates were adjusted (if necessary) to approximately 4.8 mg/ml using their corresponding buffers. 1.0-ml aliquots of each diluted sample were lyophilized immediately. The remaining sample was stored at 5° C. for 3 days and then placed at −80° C. or 50° C. Lyophilized samples were stored at 5° C. for 2 days and then either reconstituted and placed at −80° C. (T₀) or placed at 50° C. (T₇ and T₁₄). All lyophilized samples were reconstituted with 1 ml of RODI water at the appropriate time intervals. Cake appearance and reconstitution time (lyophilized samples) and RALS (liquid and lyophilized samples) were recorded at the appropriate time intervals.

For the evaluation of combinations of excipients in iteration 4, CD4-IgG2 was dialyzed into 20 mM histidine, pH 6. Filter-sterilized CD4-IgG2 dialysate was diluted to approximately 1 mg/ml to obtain the final buffer conditions shown in Table 2.

A solution comprising 20 mM histidine, 500 mM glycine was used to adjust the osmolality of buffer conditions 3 and 5 (see Table 2) closer to a target of 290 mOsm/kg. An aliquot of each solution was also prepared containing 0.05% Tween 80. A small aliquot of PRO542 in PBS (not filter-sterilized) was also prepared at 1 mg/ml with and without 0.05% Tween 80.

Evaluation of Effects of Trehalose Versus Maltose on Long-Term CD4-IgG2 Stability

CD4-IgG2 was dissolved to a concentration of 25 mg/ml and 150 mg/ml in the following seven buffer solutions: 20 mM histidine, pH 6; 250 mM glycine/20 mM histidine pH 6; 7% maltose/20 mM histidine, pH 6; 7% trehalose/20 mM histidine, pH 6; 200 mM glycine/20 mM histidine, pH 6; 6% maltose/20 mM histidine, pH 6; and 6% trehalose/20 mM histidine, pH 6. The solutions were incubated for extended periods at 4° C., −90° C., 37° C. and room temperature (about 25° C.), and the percentage purity of the samples (monomer vs. aggregate) was assessed by HPLC-SEC on a TSK column at time intervals extending up to 6 months. Results were compared to the percentage purity of a freshly dissolved sample of CD4-IgG2 (5 mg/ml) in PBS.

Evaluation of Maltose on Lyophilization of High Concentration CD4-IgG2 Solutions

The starting material, CD4-IgG2 at a concentration of 4.5 mg/ml in PBS (10 mM sodium phosphate, 140 mM sodium chloride, pH 7.0), was purified by gel filtration over Superdex® 200. WFI (water-for-injection) was used for reconstitution of formulations as well as for dilution of buffers.

Four starting buffer formulations were used: 20 mM histidine, pH 6.0; 20 mM histidine, 2% maltose, pH 6.0; 20 mM histidine, 6% maltose, pH 6.0; and 3× diluted 20 mM histidine, 6% maltose, pH 6.0 (i.e., 6.7 mM histidine, 2% maltose, pH 6.0).

The CD4-IgG2 starting material (at 4.5 mg/ml in PBS) was concentrated to 50 mg/ml in a centrifugal filter unit (Centricon Plus-80®; Millipore, Billerica, Mass.) and filtered through a 37 mm Acrodisc® syringe filter containing a GF 0.2 um Supor® membrane (Pall Corp., Ann Habor, Mich.). The concentration of CD4-IgG2 was determined by UV spectroscopy (absorbances measured at 280, 260 and 340 nm).

An aliquot of the 50 mg/ml material was left at 4° C. as a pre-lyophilization control. 1-ml CD4-IgG2 samples (50 mg/ml) were frozen in 7-ml glass vials at −80° C. overnight and lyophilized either for 5 h or overnight (˜18 h) using a 3-liter bench-top lyophilizer (VirTis, Gardiner, N.Y.). The lyophilized material was reconstituted to a concentration of 50 mg/ml by adding a volume of WFI equal to the starting volume, or to 150 mg/ml by adding one third of the starting volume of WFI. The reconstituted samples were quantified and evaluated for purity by HPLC-size exclusion chromatography on a TosoHaas TSK G3000SW_{XL} column (Tosoh Bioscience, Montgomeryville, Pa.). All samples were diluted to 0.5 mg/ml in PBS, and 100 μl aliquots were injected for analysis using a flow rate of 1 ml/min. PBS was used as the mobile phase and detection of the sample was by UV absorbance at 280 nm. The bioactivity of the samples was measured by the resonance energy transfer (RET) assay.

Concentration Study

CD4-IgG2 dissolved in PBS (5.1 mg/ml) was dialyzed into histidine buffer, pH 6. Dialyzed stock was then divided into two 30,000 molecular weight cut-off centrifuge filters and concentrated approximately 2-fold (˜26 ml to ˜13 ml each). Samples were then diluted back to the original volume in 1) histidine buffer, pH 6, or 2) histidine buffer containing 100 mM glycine, pH 6 (to bring final concentration of glycine to 50 mM). Samples were further concentrated using centrifugal force down to approximately the hold-up volume of the filter unit (200 μl). Concentrated CD4-IgG2 was evaluated for purity and concentration by HPLC-SEC immediately and after 11 and 18 days at 5° C.

TABLE 2
Buffer conditions for the combinations of excipients in iteration 4

| | | Estimated | Estimated | Final |
|---|---|---|---|---|
| | | Trehalose | Glycine | Sample |
| | | Concentra- | Concentra- | Osmolality |
| Buffer | Buffer Conditions | tion (%) | tion (mM) | (mOsm/kg) |
| 1 | 20 mM histidine | 0 | 0 | 16 |
| 2 | 50 mM histidine | 0 | 0 | 47 |
| | (estimated actual: | | | |
| | 44 mM) | | | |
| 3 | 20 mM Histidine + | 0 | 304 | 283 |
| | glycine | | | |
| 4 | 20 mM Histidine + | 2.5 | 0 | 78 |
| | trehalose | | | |
| 5 | 20 mM Histidine + | 1.8 | 244 | 273 |
| | glycine + trehalose | | | |

Real-Time Stability Evaluation

Selected formulations (see Table 3) were prepared using a process of diafiltration/concentration along with SEC on Superdex® S-200, and examined for real-time stability (up to 3 months) using biophysical assays (TSK sizing gel analysis for integrity, SDS-PAGE analysis for purity) and an antiviral bioactivity assay (RET assay).

Resonance Energy Transfer (RET) Assay

The RET assay, which measures HIV-1 envelope glycoprotein-mediated membrane fusion, was performed as described by Litwin et al. (1996). This fluorescence-based technique involves labeling one fusion partner (a cell line expressing gp120/gp4l) with the fluorescent dye, octadecyl fluorescein (F18; Molecular Probes, Eugene, Oreg.) and the other fusion partner (a CD4-expressing T cell line) with octadecyl rhodamine (R18; Molecular Probes). These probes consist of fluorescent molecules conjugated to 18-C saturated hydrocarbon chains that spontaneously insert into cell membranes without inhibiting cellular replication or fusion efficiency. The emission spectrum of F18 overlaps with the excitation spectrum of R18, allowing fluorescence RET to occur when the dyes are brought into close association in the same membrane following fusion. The RET signal is directly related to the amount of HIV-1-mediated membrane fusion, and is quantified by exciting F18 at 450 nm and measuring the R18 emission at 590 nm.

Pharmacotoxicological and Pharmacokinetic Studies

Pharmacotoxicological and pharmacokinetic studies were conducted in rabbits with selected “high-concentration” and control CD4-IgG2 formulations (see Table 3). Formulations 2 and 3 were constituted to meet appropriate osmolality for animal studies, and were prepared by terminal formulation, i.e., glycine and Tween 80® were added to the required concentration to the base formulation of 20 mM histidine, pH 6. More recently, an alternative method of preparing formulations by direct concentration into a formulation containing 20 mM histidine and 200 mM glycine, pH 6 has been developed. These latter formulations have been made at a concentration of 100 mg/ml. As used herein, “high concentration” formulations of CD4-IgG2 refer to formulations in which the concentration of CD4-IgG2 ranges from about 100-162 mg/ml. It should be noted that this invention also describes novel formulations that contain higher concentrations (25-100 mM) of CD4-IgG2 than were used previously (<10 mg/ml) though these are not covered by the term “high concentration” formulations.

Three subsets (A, B and C), each containing three groups of three male New Zealand White rabbits were used in the comparative study (except for Group 3 of Subset A which contained two males). Animals in Subset A were administered CD4-IgG2 in the left ear as a single intravenous bolus injection of 3.0 ml (at 5 mg/ml) for Group 1 and 110 μl (at 140 mg/ml) for Groups 2 and 3. Animals in Subset B were administered CD4-IgG2 in the left sacrospinal muscle as divided intramuscular injections of 1.5 ml each (3.0 ml total) for Group 1, and single injections of 110 μl for Groups 2 and 3. The divided doses for Formulation 0 were between one site on the left and one site on the right. Formulations 2 and 3 were dosed at a single site on the left. Animals in Subset C were administered CD4-IgG2 in the nape of the neck as divided subcutaneous injections of 1.5 ml each (3.0 ml total) for Group 1 and single injections of 110 μl for Groups 2 and 3. The divided doses for Formulation 0 were split between one site on the left and one site on the right. Formulations 2 and 3 were dosed at a single midline site.

TABLE 3
Formulations used for real-time stability evaluation
and pharmacokinetic study

| Formulation | Composition |
|---|---|
| 0 | 5 mg/ml CD4-IgG2 in PBS |
| 1 | 150 mg/ml CD4-IgG2 in 20 mM His, pH 6 |
| 2 | 140 mg/ml CD4-IgG2 in 20 mM His, 200 mM Gly, pH 6 |
| 3 | 140 mg/ml CD4-IgG2 in 20 mM His, 200 mM Gly, |
| | 0.05% Tween 80, pH 6 |

All sites were marked with an indelible marker at the area of entry of the needle. Intravenous sites were also marked approximately at the end of needle progress and approximately 1 cm from that point. The areas beyond the point of entry of the needle and its progress were evaluated macroscopically and microscopically. All animals were sacrificed via lethal barbiturate injection into the right ear on day 15.

Clinical observations for pharmacotoxicological signs and/or mortality were recorded at 1, 4, 24, 48 and 72 h post-dose and daily through to day 15. Injection site observations were scored at 1, 24 and 72 h and on days 8 and 15 using the Draize evaluation score (Draize, 1959). Body weights were recorded pre-test, on the day of dosing (day 1), and on days 8 and 15.

Serial blood samples (˜500 μl each) were collected from-the ear vein at approximately ˜5 min, 1, 2, 4, 8 and 18 hours post-dose, and on days 2, 3, 5, 8, 11 and 15 post-dose from each animal. Serum samples were obtained by centrifugation and transferred into prelabeled plastic cryotubes. Serum samples were stored frozen at approximately −70° C.±10° C. prior to determining the plasma-levels of the drug by ELISA. At necropsy, each rabbit in Subset A (IV) was administered 1% Evans Blue via the left marginal ear vein ˜30 minutes prior to terminal sacrifice. At sacrifice, the left ear (CD4-IgG2-treated) of each rabbit in Subset A was excised, flushed with normal saline and observed under a dissecting microscope to record the intensity of dye staining. A full-thickness section of the pinna (about 6×6 cm), including the artery and injection site, was removed and fixed in 10% neutral buffered formalin for histopathological evaluation. Representative portions of all injection sites (IV, IM and SC) were collected for evaluation and placed in 10% neutral buffered formalin along with selected organs for possible histopathological analysis.

Results and Discussion

Profile of structural stability of CD4-IgG2 An initial study was undertaken to obtain a profile of the structural stability of CD4-IgG2 against changes in variables including freeze/thaw cycling, pH, temperature, ionic strength and shear stress. The intent was to map the strengths and weaknesses of the CD4-IgG2 molecule in a base buffer (BB) as well as in the PBS buffer originally used to constitute the protein solution. Stability profiling also allowed for the identification of a molecule-specific screening process which was subsequently used to develop formulation matrices for improved stability of CD4-IgG2.

In the ANS titration done as a prelude to the EF analyses, the 520/490 nm emission ratio reached a plateau after adding ANS to a concentration of 0.11 mM. This ANS concentration was used in all subsequent EF analyses. In the guanidine titration, maximum sensitivity was achieved when the emission was monitored at a ratio of 358 nm to 318 nm. This ratio was monitored in all subsequent IF measurements. The maximum ratio observed upon complete unfolding of the protein by guanidine was ˜3.2. This information was useful in evaluating the IF data.

Successive freeze/thaw cycles caused the total protein recovered to decrease by ˜6% per cycle over the first two cycles, and by ˜10-20% per cycle over subsequent cycles. It was visually observed that the samples had increased amounts of precipitation with each successive cycle of freeze/thaw. The percentage purity of the CD4-IGG2 peak was found to range from 93% (1 and 2 freeze/thaws) to 91% (6 freeze/thaws). These data suggested that freeze/thawing of samples causes the CD4-IgG2 to precipitate out of solution and that the originally constituted CD4-IGG2 could withstand no more than 2 freeze/thaw cycles if significant loss of protein by aggregation and precipitation were to be avoided.

To evaluate the effects of ionic strength, samples in BB containing 0, 10, 100 and 500 mM NaCl and a sample in PBS were incubated at 50° C. for 0, 3, 7 or 14 days and analyzed by a battery of biophysical tests described previously (see Methods). In the presence of 100 and 500 mM NaCl, recovery of the protein monomer as assessed by HPLC-SEC was about 60% and 35% after 7 and 14 days respectively, compared to about 20% for both time periods in the absence of NaCl, and about 45% and 28% after 7 and 14 days respectively in the presence of 10 mM NaCl. UV spectroscopy also showed increased protein recovery with increased NaCl content. Thus, addition of NaCl increases the percentage of monomer recovery. The purity of CD4-IgG2 was lower in high NaCl solutions; for example, the purity was about 100% and 93% after 14 days in the absence of NaCl and in the presence of 10 mM NaCl respectively, but dropped to about 52% and 22% after 14 days in the presence of 100 mM and 500 mM respectively. However, this apparent drop in purity could be due to solubilization of high molecular weight species in the high salt formulations, resulting in a greater proportion of high molecular weight species compared to the monomer. At day 21, 0 and 10 mM NaCl samples were cloudy while PBS, 100 and 500 mM NaCl samples were clear with small precipitates. This indicated that the protein more readily precipitates out of solution in low salt buffers, and is consistent with the quantitative data that protein recovery was lower in these buffers.

A RALS analysis of CD4-IgG2 in buffers containing 0-100 mM NaCl at time zero showed that the transition temperature at different ionic strengths is not significantly different but that the initial value before the temperature stress exhibited some ionic strength dependency. Generally, as NaCl concentration increased, the initial RALS reading tended to decrease indicating decreased aggregation. The RALS data indicated that the thermal transition temperature of CD4-IgG2 is ˜56-58° C. Using this information, an accelerated short-term stability study temperature of 50° C. was determined and continued to be used for all subsequent short-term stability studies.

Shear stress and a “pH titration” stress both caused significant loss of protein. There appeared to be a trend of increasing percentage recovery after shear stress with increasing NaCl concentration which suggests a stabilizing effect by NaCl. The RALS data showed some variations with changes in ionic strength after pH stress but there was no obvious trend.

To evaluate the effect of pH, samples in base buffer and approximately 25 mM NaCl at pH's ranging from 4 to 8 and a sample in PBS were incubated at 50° C. for 0, 3 or 7 days and analyzed. All samples showed some degree of suspended precipitate at day 7. On days 3 and 7, samples at pH 7.5 and 8 were cloudy with precipitate. HPLC-SEC analysis showed that samples in buffers at pH 6 and 6.5 yielded the highest recoveries (-80%) of CD4-IgG2 monomer at day 7 compared to -25-55% recovery at other pH values. The RALS data suggested that the optimal pH range for CD4-IgG2 was 5.5-7. Measurement of EF showed that pH 6 overall exhibited the highest emission ratios, indicating less hydrophobicity and less denaturation. Thus, the optimal pH for maintaining protein stability was determined by apparent hydrophobicity analysis to be pH 6. However, subjecting samples to shear stress and measuring CD4-IgG2 monomer recovery by HPLC-SEC, recovery of total protein by UV spectroscopy, and turbidity by RALS, suggested that CD4-IgG2 could best withstand shear stress at pH 6.5.

A Pymal analysis was done to assay for the presence of free sulfhydryl groups using CD4-IgG2 samples dissolved in PBS and samples dissolved in buffer containing 1% SDS or 6 M guanidine. CD4-IgG2 in PBS showed significant emissions at both 374 and 394 nm, suggesting the presence of free-sulfhydryl groups. In the presence of 1% SDS, the fluorescence emissions were off-scale within 15 minutes (only the data point at time zero was on-scale). This suggests that there are additional free sulfhydryl groups buried within the CD4-IgG2 native conformation which are exposed upon denaturation with SDS. Denaturation with 6 M guanidine caused a ˜6-fold increase in fluorescence emissions but this was much less that the increase caused by denaturation with SDS, suggesting that internal sulfhydryl groups in the CD4-IgG2 protein are less accessible after guanidine denaturation than after SDS denaturation. That guanidine does denature the protein was evidenced by the guanidine titration/IF assay.

Overall, the stability profiling data suggested that the molecular integrity of CD4-IgG2 is susceptible to changes in temperature, ionic strength, pH and shear stress. The freeze/thaw analyses suggested that CD4-IgG2 stock material at 0.2 mg/ml in PBS buffer could be frozen and thawed up to two times without significant changes in the molecule. The addition of 100-500 mM NaCl appeared to increase the percentage recovery of CD4-IgG2 after shear and heat stress. Based on the HPLC-SEC and visual observation data, it is possible that increased NaCl concentration in heat-stressed samples tends to solubilize the molecule, especially the high molecular weight species. The thermal transition temperature of the molecule was determined to be ˜56-58° C. This means that 50° C. was an appropriate accelerated stability study temperature because it is below the molecule's transition temperature. Based on the pH evaluation data, the optimal pH appeared to be pH 6 or 6.5. The Pymal data suggested the presence of free sulfhydryl groups present in CD4-IgG2, which can cause molecular aggregation during long-term storage. Lyophilization is one method of minimizing such molecular aggregation.

Formulation Matrix Development

Four iterations of formulation development were performed to arrive at the lead buffer matrix for CD4-IgG2. In the first iteration to evaluate buffer systems, CD4-IgG2 was dialyzed into 20 mM citrate or histidine buffers ranging in pH from 5.5 to 6.5. Practically no CD4-IgG2 was recovered from T₇ samples in citrate buffers at pH 5.5 or 6.0. The absence of CD4-IgG2 after 7 days at 50° C. in both citrate buffers was probably due to the complete precipitation of the protein because the supernatants after centrifugation had base-line A₂₈₀ nm readings. Additionally, the citrate samples were observed to be cloudy after 2 days at 50° C. Among the histidine buffers, the highest recovery of CD4-IgG2 monomer, as assessed by HPLC-SEC, was at pH 6.5, suggesting that this pH is optimal for storage of CD4-IgG2. The A₂₈₀ data supported the HPLC data in that overall, the histidine buffers showed significantly greater recovery than the citrate buffers and PBS. All samples evaluated (with the exception of the T₇ samples in sodium citrate buffers) consisted of 97% or greater of monomer.

RALS readings in citrate buffers and PBS were significantly higher than those in histidine buffers, suggesting greater intermolecular interactions in the former buffers particularly after heating at 50° C. Histidine, pH 6 buffer appeared to be optimal, giving lower RALS readings than samples in histidine pH 5.5 and 6.5 buffers after heat stress (days 7 and 14).

The recovery of CD4-IgG2 monomer after shear stress was measured by HPLC-SEC. The highest recovery was obtained in pH 6.0 buffer, suggesting that CD4-IgG2 is slightly more stable in histidine buffer at pH 6 than at either pH 5.5 or 6.5. Thus, whereas the heat stress HPLC data suggested that pH 6.5 was optimal, the shear stress data suggested that pH 6.0 was optimal. This small difference between pH 6 and 6.5 are believed to be within the methods' precision. Overall, the data suggested that CD4-IgG2 is most stable in histidine buffers at pH 6.0-6.5 and very unstable in citrate buffers.

In the second iteration of formulation development, the effect of amino acid stabilizers was evaluated. The recovery of CD4-IgG2 monomer after incubation at 50° C. for 0, 7 and 10 days was measured by HPLC-SEC. Setting arbitrary cut-off points of 85% and 74% recovery for T₇ and T₁₄ samples respectively included CD4-IgG2 in formulation matrices containing histidine without stabilizer, and histidine with alanine, glycine, and glycylglycine. All formulation matrices containing histidine showed greater recovery than the sample in PBS. Setting an arbitrary cut-off point of 65% for percentage purity of T₁₄ samples included formulation matrices containing histidine without stabilizer, and histidine with alanine, glycine, proline, and glycylglycine. The remaining percentages of the samples consisted of high molecular weight species for all histidine samples. The PBS control sample was the only one to show low molecular weight species (2.5% at T₇ and 9% at T₁₄) in addition to high molecular weight species. All the sample solutions remained clear over 7 days at 50° C. except for the PBS sample which became very cloudy.

Based on UV absorbance at 280 nm, most samples showed >100% recovery over both 7- and 14-day periods with the exception of the PBS control sample which showed ˜40% and ˜10% recovery after 7 and 10 days respectively. An increase in high molecular species present in the T7 and T₁₄ histidine samples may have been responsible for the increase in the UV absorbance compared to the To sample, probably reflecting a different extinction coefficient for the high molecular weight species. The low percentage recoveries in the PBS samples were consistent with the HPLC-SEC results and the visual observation of cloudiness apparently attributable to precipitation of the protein.

Some discrepancies in total protein concentrations as measured by A₂₈₀ were evident compared to the monomer protein concentrations as measured by SEC-HPLC, especially for arginine and lysine. These are likely attributable to the presence or absence of high molecular weight species. Based on the percentage recovery and purity estimated by HPLC, the percentage recovery estimated by UV spectrophotometry, and the visual observations, it appeared that over time and with heat stress the histidine samples formed soluble, high molecular weight species. By contrast, the PBS control samples formed high and low molecular weight species which were mostly insoluble.

RALS analysis of 7-day, temperature-stressed samples suggested that the presence of creatinine, proline or glycylglycine caused a reduction in protein-protein interaction (aggregation).

Samples were shear stressed for 4 hours (T₀ samples) or 2.5 hours (T₇ samples) and analyzed by A₂₈₀ measurement, HPLC-SEC and RALS. The percentage recovery as assessed by A₂₈₀ and by HPLC-SEC after shear stress generally followed the same trend seen without shear stress. The percentage purity of the To samples before and after shear stress was high (≧97% except for isoleucine and leucine which were 90-93%). The chromatograms showed that most of the CD4-IgG2 was present as monomer with the remaining amount (1-10%) present as high molecular weight species. Arbitrary cut-offs were assigned to the data for the percentage recovery and purity after shear stress in an effort to screen out stabilizers. CD4-IgG2 in formulation matrix containing 20 mM histidine, pH 6 without stabilizer and containing alanine, glycine or proline were the leading candidates from this set of data. The PBS control formulation performed comparable to these leading candidates.

RALS measurements on shear-stressed T₇ samples suggested that the presence of creatinine, proline, glycine, isoleucine or leucine is beneficial for protection against shear stress. In contrast to the T₀ shear stress results, the 7-day heat-stressed PBS control sample performed significantly worse than the histidine-based samples.

The percentage recovery after shear stress was overall high in all cases (88-104%). Glycine and alanine showed slightly higher percentage recoveries than the other histidine-based formulations. Even though the percentage recoveries for arginine, lysine, and PBS were high after shear stress (104% for the PBS control sample), the actual monomer concentrations were low, which excludes them as lead matrices.

Shear stress data showed percentage recoveries mostly exceeding 85% but adjustment of experimental conditions to achieve approximately 50% average shear stress recovery was deemed useful in trying to identify lead matrices. Therefore, to optimize the shear stress excipient screening technique, an experiment was run in which additional shear time (20-24 h) was applied to a representative stabilizer evaluation sample containing 20 mM histidine plus 50 mM leucine.

The results of HPLC-SEC and RALS analyses as well as the visual appearance of samples after 20-24 h of shear stress suggested that CD4-IgG2 in histidine-based formulation matrix is not very sensitive to shear stress. The stressed samples appeared cloudy but showed slightly increased monomer percentage purity, suggesting that the cloudiness was due to precipitation of high molecular weight species. Although the temperature stress at 50° C. provided information to proceed further with the selection of lead matrices, the 20-24 h shear stress data also suggested that an additional stress might be essential to be included in the excipient screening process to further discriminate between different formulation matrices. Hence pH stress was used to evaluate a representative stabilizer-containing sample.

CD4-IgG2 in arginine-containing formulation matrix was chosen for analysis because this sample exhibited temperature-dependent instability and hence was thought more likely than others to demonstrate instability from pH stress. After pH stress, all samples (CD4-IgG2 in histidine buffer only; histidine plus arginine; arginine only; and PBS buffer) decreased in total protein and monomer concentration and in percentage monomer species, and RALS readings increased significantly, indicating protein aggregation. All stressed samples, except for the one in histidine pH 6 buffer, also appeared cloudy. These data revealed that pH does affect CD4-IgG2 stability in the formulation matrices tested. This method was able to distinguish between histidine versus histidine plus arginine buffers, consistent with previous results (histidine optimal compared to histidine plus arginine), and thus has potential use as an excipient-screening tool.

The third iteration of formulation development involved evaluating the effect of carbohydrates that could serve as stabilizers, bulking agents and/or lyoprotectants. Carbohydrate samples (both liquid form and reconstituted lyophilized form) were analyzed with and without heat stress and with shear stress. As expected, lyophilized samples generally showed increased recovery compared to the corresponding liquid samples, indicating increased stability. Sucrose, maltose and trehalose gave favorable recovery and purity results in the various assays. Based on the heat and shear stress tests conducted over a 2-week period, trehalose was considered the lead matrix because its percentage recoveries for both liquid and lyophilized T₁₄ samples were among the highest obtained. However, in longer-term stability testing over up to 6 months (see below), maltose proved to be a more effective stabilizing agent than trehalose.

Purity was generally high for the heat-stressed samples through day 14. It was expected that percentage recovery of T₇ samples would be greater than T₇ stress samples and T₁₄ samples, but the data did not show this in all cases, especially with the lyophilized samples. Method inaccuracies and, in the lyophilized samples not taking exact water evaporation into account, may account for the discrepancies.

RALS readings were taken on all liquid and reconstituted lyophilized samples at T₀, T₇ and T₁₄ (before freezing for later analysis). RALS readings for the lyophilized samples (after reconstitution) were higher than the liquid samples with the exceptions of sucrose and maltose. Undissolved lyophilized particles were possibly the cause of the slightly higher RALS readings in the lyophilized samples. The PBS control formulation had a much higher RALS intensity, suggesting a higher degree of aggregation, than the histidine-based samples. This observation was confirmed visually as both the liquid and the reconstituted lyophilized PBS samples were cloudy whereas the histidine-based samples were clear.

In most liquid samples, the RALS intensity increased from day 7 to day 14, suggesting aggregation over time with heat stress. In the lyophilized samples, by contrast, the RALS intensity decreased or remained approximately the same over time. With the exception of the PBS sample, the RALS intensities of the lyophilized samples were lower than those of the liquid samples. These observations suggest greater stability in the lyophilized samples than the liquid samples after heat stress.

For the T₀ lyophilized samples, a white cake was formed which dissolved immediately except for the fructose sample which dissolved within ˜2 min and the PBS sample which required 5-10 min to dissolve and which remained cloudy after reconstitution. The cake appearance of the lyophilized samples remained generally similar over days 7 to 14. For the T₇ and T₁₄ lyophilized samples, a white cake was formed which dissolved immediately except for the sorbitol and fructose samples which took 2-6 min for dissolution.

The fourth iteration of formulation development evaluated a combination of excipients. SEC-HPLC was performed on samples with various combinations of excipients (see Table 2 for combinations used) with and without heat stress and with shear stress, and the results are summarized in Table 4.

Buffer matrices containing Tween-80® generally showed increased CD4-IgG2 monomer recovery. The combination containing histidine, glycine, Tween 80 and trehalose was considered the lead formulation because it most frequently gave the highest percentage recovery.

The percentage purity was higher for Tween-minus samples compared to the corresponding Tween-added samples. However, Tween addition was deemed beneficial due to the increased recovery obtained with the Tween-added samples. Furthermore, the apparent lower purity in Tween-added samples may be misleading. It is possible that Tween increases solubility of high molecular weight species, resulting in an apparent percentage decrease in the purity of the monomeric species.

TABLE 4
Percentage monomer recovery from T₀, T₇ and T₁₄
samples with combinations of excipients

| | T₀ | | T₇ | |
|---|---|---|---|---|
| Buffer Conditions | Stress | T₇ | Stress | T₁₄ |
| 20 mM histidine | 52 | 67 | 30 | 74 |
| 50 mM histidine | 0 | 56 | 4 | 51 |
| 20 mM histidine + glycine | 42 | 86 | 55 | 78 |
| 20 mM histidine + trehalose | 47 | 75 | 53 | 77 |
| 20 mM histidine + glycine + trehalose | 20 | 84 | 13 | 79 |
| phosphate-buffered saline, pH 7 | | | | |
| 20 mM histidine + Tween | 66 | 71 | 55 | 70 |
| 50 mM histidine + Tween | 84* | 57 | 37 | 51 |
| 20 mM histidine + glycine + Tween | 75 | 86 | 61 | 78 |
| 20 mM histidine + trehalose + Tween | 45 | 82 | 66 | 76 |
| 20 mM his + gly + trehalose + Tween | 81 | 90 | 68 | 85 |
| phosphate-buffered saline + Tween | | 0 | | 0 |

*For each treatment, the highest percentage recoveries are highlighted in bold font.

To determine feasibility of achieving the target CD4-IgG2 concentration of 150 mg/ml that is suitable for SC and IM administration, a concentration study was performed. CD4-IgG2 was concentrated to ˜150 and ˜162 mg/ml by centrifugal filtration, stored at 5° C. for 0, 11 and 18 days, then diluted ≧200-fold to ˜0.8 μg/μl and analyzed by HPLC-SEC. CD4-IgG2 at ≧150 mg/ml in histidine ±50 mM glycine, pH 6 appeared generally stable with a percentage recovery of monomer of ˜100% after 11 days and increasing to ˜140% after 18 days storage at 5° C. The apparent increase in concentration over time is probably due to inaccuracy of the method used for estimating protein concentration as this was not optimized for samples with elevated protein concentrations. The purity of the recovered CD4-IgG2 was ≧95%, the 2-5% impurity consisting of high molecular weight species.

In conclusion, histidine buffer at pH 6 was chosen from the first iteration of formulation development as the lead buffer matrix. From the second iteration, histidine buffer, pH 6±50 mM glycine were selected as the lead matrices. From the third iteration, histidine buffer, pH 6 containing 3% trehalose was selected as the lead matrix. The data suggested that the stabilities of CD4-IgG2 in histidine-based formulations were significantly greater than in the original formulation in PBS. Lyophilization of CD4-IgG2 at 5 mg/ml was demonstrated and lyophilized samples generally showed greater percentage monomer recovery than the liquid form. Based on the combined data from iterations 1 through 4, a formulation comprising 20 mM histidine, glycine and Tween 80 adjusted to pH 6 appeared to be the optimal buffer matrix for a CD4-IgG2 150 mg/ml liquid formulation. For lyophilization, a formulation comprising 20 mM histidine, glycine, trehalose and Tween 80 adjusted to pH 6 appeared to be optimal.

Real-Time Stability of High-Concentration CD4-IgG2 Formulations

Formulations 1, 2 and 3 (see Table 3) were analyzed by TSK size exclusion chromatography and SDS-PAGE to evaluate real-time stability. Formulation 1 was stable at 4° C. for up to 3 months (97-99% intact) and Formulation 2 was stable for up to 2 months (97% intact). Formulation 3 was stable for up to 2 months (93.6% intact). The more recently developed 100 mg/ml, direct concentration formulation was found by TSK sizing analysis to be stable at 4° C. and −90° C. for up to 6 weeks (98% intact).

The CD4-IgG2 formulations 0-4 (see Table 3) were tested using the RET assay for their ability to inhibit HIV-1 envelope-mediated cell membrane fusion, and the results are shown in Table 5. IC₅₀ is the concentration required to inhibit fusion by 50%. These data show that the IC₅₀ values in the high-concentration formulations are close to the PBS control values, thereby establishing that all the formulations possess good anti-viral activity (as measured by inhibition of HIV-1 envelope glycoprotein-mediated membrane fusion).

TABLE 5
Results of RET assay

| | | |
|---|---|---|
| Formulation* | IC₅₀ (μg/ml) | IC₉₀ (μg/ml) |

Month 1

| | | |
|---|---|---|
| 0 | 0.248 | 0.837 |
| 1 | 0.294 | 0.945 |

Month 2

| | | |
|---|---|---|
| 0 | 0.450 | 1.306 |
| 1 | 0.533 | 1.470 |
| 2 | 0.522 | 1.469 |
| 3 | 0.578 | 1.571 |

Month 3

| | | |
|---|---|---|
| 0 | 0.831 | 3.534 |
| 1 | 1.253 | 17.194 |
| 2 | 1.442 | 10.641 |
| 3 | 1.207 | 4.711 |

Month 4

| | | |
|---|---|---|
| 0 | 0.652 | 2.353 |
| 1 | 0.399 | 1.359 |
| 2 | 0.514 | 1.631 |
| 3 | 0.556 | 1.763 |

*See Table 3 for composition of each formulation.

Effectiveness of Maltose in Enhancing Stability of CD4-IgG2 in Liquid Media

The stability of low and high concentration formulations of CD4-IgG2 in maltose- versus trehalose-containing solutions was compared at −90° C., 4° C., room temperature (˜25° C.) and 37° C. over extended periods of up to 6 months. The results are summarizared in Tables 6-9.

TABLE 6
Percentage purity of CD4-IgG2 stored in liquid
formulations at −90° C.

| Solutions | Wk 1 | Wk 2 | Mo 1 | Mo 2 | Mo 3 | Mo 4 | Mo 5 | Mo 6 |
|---|---|---|---|---|---|---|---|---|
| 25 mg/ml | | | | | | | | |
| CD4-IgG2 in | | | | | | | | |
| 20 mM His | 97.9 | 96.6 | 97.1 | 97.3 | 98.5 | 97.3 | 96.9 | 99.9 |
| 250 mM Gly/ | 97.6 | 97.1 | 98.5 | 97.8 | 97.6 | 97.3 | 97.6 | 99.2 |
| 20 mM His | | | | | | | | |
| 7% Maltose/ | 97.7 | 96.8 | 97.9 | 97.0 | 97.0 | 98.4 | 97.1 | 98.9 |
| 20 mM His | | | | | | | | |
| 7% Trehalose/ | 95.6 | 94.8 | 95 | N/A | N/A | N/A | N/A | N/A |
| 20 mM His | | | | | | | | |
| 150 mg/ml | | | | | | | | |
| CD4-IgG2 in | | | | | | | | |
| 20 mM His | 94.3 | 94.2 | 95.4 | 95.4 | 96.3 | 94 | 93.9 | 96.4 |
| 200 mM Gly/ | 94.96 | 95.3 | 96.2 | 96.2 | 96.5 | 94.6 | 94.6 | 96.7 |
| 20 mM His | | | | | | | | |
| 6% Maltose/ | 95.9 | 95.9 | 96.3 | 96.3 | 96.9 | 95.3 | 96.3 | 97.0 |
| 20 mM His | | | | | | | | |
| 6% Trehalose/ | 94.5 | 95.9 | 95.0 | N/A | N/A | N/A | N/A | N/A |
| 20 mM His | | | | | | | | |

His = histidine;
Gly = glycine;
all solutions are at pH 6.0
Wk = week;
Mo = month;
N/A = not analyzed

TABLE 7
Percentage purity of CD4-IgG2 stored in liquid formulations at 4° C.

| Solutions | Day 0 | Wk 1 | Wk 2 | Mo 1 | Mo 2 | Mo 3 | Mo 4 | Mo 5 | Mo 6 |
|---|---|---|---|---|---|---|---|---|---|
| 25 mg/ml CD4-IgG2 in | | | | | | | | | |
| 20 mM His | 99.3 | 97.4 | 96.5 | 98.7 | 97.7 | 97.4 | 97.5 | 97.2 | 98.2 |
| 250 mM Gly/20 mM His | 99.6 | 98.0 | 98.0 | 98.4 | 98.1 | 98.3 | 98.6 | 98.0 | 98.5 |
| 7% Maltose/20 mM His | 97.9 | 97.3 | 97.5 | 97.8 | 98.8 | 98.4 | 98.3 | 98.5 | 98.7 |
| 7% Trehalose/20 mM His | 95.5 | 95.9 | 94.9 | 94.8 | N/A | N/A | N/A | N/A | N/A |
| 150 mg/ml CD4-IgG2 in | | | | | | | | | |
| 20 mM His | 95.5 | 94.3 | 93.9 | 93.2 | 91.6 | 90.9 | 89.6 | 89.7 | N/A |
| 200 mM Gly/20 mM His | 95.4 | 96.1 | 94.4 | 94.0 | 93.8 | 95.3 | 93.1 | 96.3 | 91 |
| 6% Maltose/20 mM His | 96.7 | 96.4 | 95.4 | 95.5 | 94.8 | 96.0 | 94.3 | 98.8 | 64** |
| 6% Trehalose/20 mM His | 94.1 | 93.0 | 92.3 | 87.4 | N/A | N/A | N/A | N/A | N/A |

**Mold grew in this vial
His = histidine;
Gly = glycine;
all solutions are at pH 6.0
Wk = week;
Mo = month;
N/A = not analyzed

TABLE 8
Percentage purity of CD4-IgG2 stored in liquid
formulations at room temperature (25° C.)

| Solutions | Week 1 | Week 2 | Month 1 | Month 2 | Month 3 | Month 4 |
|---|---|---|---|---|---|---|
| 25 mg/ml CD4-IgG2 in | | | | | | |
| 20 mM His | 98.9 | 97.6 | 97.3 | 96.0 | 96.8 | 96.4 |
| 250 mM Gly/20 mM His | 98.8 | 97.2 | 98.5 | 96.9 | 97.2 | 96.9 |
| 7% Maltose/20 mM His | 98.4 | 97.5 | 98.1 | 96.7 | 97.2 | 97.4 |
| 7% Trehalose/20 mM His | 95.2 | 92.1 | 76.3 | N/A | N/A | N/A |
| 150 mg/ml CD4-IgG2 in | | | | | | |
| 20 mM His | 93.9 | 90.4 | 90.4 | 84.2 | N/A | N/A |
| 200 mM Gly/20 mM His | 95.3 | 92.6 | 91.7 | 87.4 | N/A | N/A |
| 6% Maltose/20 mM His | 95.5 | 94.7 | 93.0 | 91.8 | N/A | N/A |
| 6% Trehalose/20 mM His | 89.6 | 76.7 | N/A | N/A | N/A | N/A |

His = histidine;
Gly = glycine;
all solutions are at pH 6.0
Wk = week;
Mo = month;
N/A = not analyzed

TABLE 9
Percentage purity of CD4-IgG2 stored in liquid formulations at 37° C.

| Solutions | Week 1 | Week 3 | Month 1 | Month 2 |
|---|---|---|---|---|
| 25 mg/ml CD4-IgG2 in | | | | |
| 20 mM His | 95.2 | 93.2 | 91.5 | 85.9 |
| 250 mM Gly/20 mM His | 96.5 | 95.2 | 93.0 | 87.2 |
| 7% Maltose/20 mM His | 96.8 | 95.9 | 93.7 | 89.1 |
| 7% Trehalose/20 mM His | 78.0 | 54.3 | N/A | N/A |
| 150 mg/ml CD4-IgG2 in | | | | |
| 20 mM His | 65.9 | 54.7 | N/A | N/A |
| 200 mM Gly/20 mM His | 77.5 | 67.8 | N/A | N/A |
| 6% Maltose/20 mM His | 80.1 | 72.3 | N/A | N/A |
| 6% Trehalose/20 mM His | 39.7 | degraded | N/A | N/A |

His = histidine;
Gly = glycine;
all solutions are at pH 6.0
N/A = not analyzed

A freshly dissolved solution of CD4-IgG2 (5 mg/ml) in PBS consistently showed a purity of 98.2%. At −90° C., maltose did not have a significant effect on the stability of CD4-IgG2 over 6-month storage period since the protein showed high stability in 20 mM histidine, pH 6.0, without any supplements, and in 20 mM histidine, 250 mM glycine, pH 6.0. Trehalose, present at 6-7%, appeared to reduce stability slightly though trehalose-containing samples were only tested over a 1-month period.

At 4° C., a slight stabilizing effect due to maltose was observed, and trehalose again had a deleterious effect on CD4-IgG2 stability, with a more pronounced effect evident with the high-concentration formulation of CD4-IgG2 (150 mg/ml). These effects were amplified at higher temperatures. For example, at room temperature, the 25 mg/ml CD4-IgG2 solution in the presence of 7% trehalose showed only 76.3% purity after 1 month whereas the 150 mg/ml sample showed 76.3% purity after only 2 weeks. By comparison, in solutions containing 6-7% maltose the recorded purity was 98.1% and 94.7% after 1 month and 2 weeks, respectively. At 37° C., the 150 mg/ml CD4-IgG2 solution in the presence of 6% trehalose showed only 39.7% purity after 1 week and was degraded after 2 weeks. The corresponding 150 mg/ml CD4-IgG2 sample in the presence of 6% maltose showed 80.1 and 72.3% purity after 1 and 2 weeks respectively at 37° C. These levels of purity were higher than for samples stored in 20 mM histidine buffer, pH 6.0, or in 20 mM histidine buffer, 200 mM glycine, pH 6.0. It was concluded, therefore, that although a formulation comprising 20 mM Histidine, pH 6.0, glycine, trehalose and Tween 80 appeared to be optimal for lyophilization of CD4-IgG2 solutions, the presence of maltose in place of trehalose appreciably enhances the stability of CD4-IgG2 stored in solution at room temperature or higher.

Lyophilization of CD4-IgG2 in maltose-containing solutions In view of the stabilizing effect of maltose on solutions of CD4-IgG2, the efficacy of maltose as a lyoprotectant was also evaluated. After lyophilization, the white cake that was formed in the vial took no more than 30 min to disappear after reconstitution and the solution looked clear.

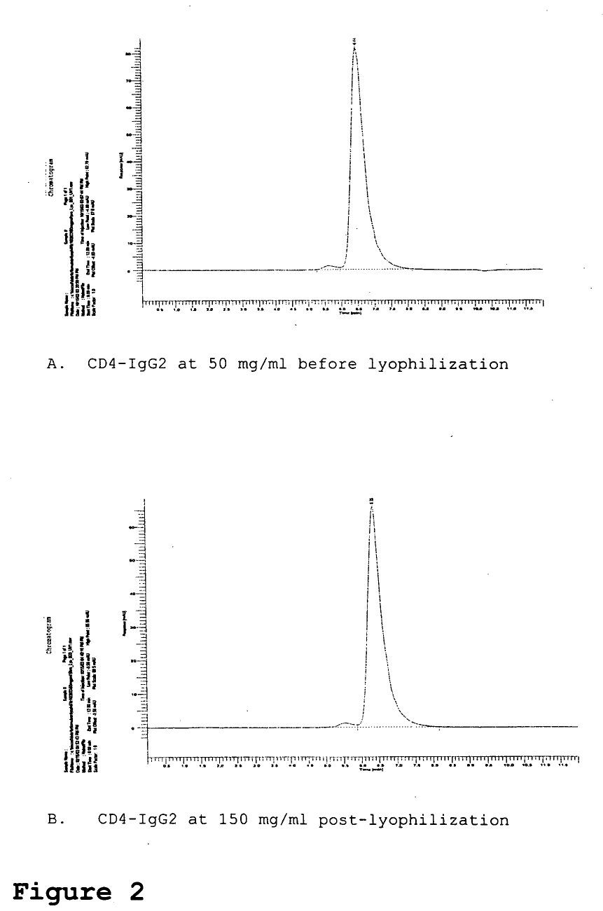

TSK analyses (Table 10) showed that 3 to 5% aggregates were detectable post-lyophilization in both 50 mg/ml and 150 mg/ml formulations when maltose was omitted from the starting buffer. It was observed that a higher level of aggregates (>4%) was present when 6% maltose was added to the 150 mg/ml formulation post-lyophilization. By contrast, <1% aggregates were detected for both the 50 mg/ml and 150 mg/ml formulations when maltose was present in the starting buffer prior to lyophilization (see FIG. 2 and Table 10). Lyophilization, carried out either for 5 h or overnight, resulted in a percentage purity of 98.3% and 97.4%, respectively (Table 10), suggesting that a shorter lyophilization time was preferable. In addition, these data also suggest that 150 mg/ml may be close to the upper limit for the reconstitution/formulation of CD4-IgG2.

The results of RET analysis of the lyophilized samples (Table 11) showed that both the reconstituted 50 mg/ml and 150 mg/ml formulations had similar bioactivity after lyophilization as compared to a non-lyophilized, liquid control.

It was concluded that an efficacious way to prepare a high-concentration CD4-IgG2 formulation is to first concentrate it to about 50 mg/ml in a buffer comprising about 6.7 mM histidine/2% maltose, pH 6.0, then lyophilize the sample and reconstitute it to about 150 mg/ml in a buffer comprising about 20 mM histidine/6% maltose, pH 6.0. The reconstituted lyophilized 150 mg/ml formulation has been shown to exhibit a very low level of aggregates (<1%) and a level of bioactivity equivalent to that of a reference liquid control.

TABLE 10
TSK analyses (HPLC-SEC) of CD4-IgG2 lyophilized in
histidine/maltose buffer

| | | | CD4- | | |
|---|---|---|---|---|---|
| Start | | | IgG2 | End | |
| concen- | Start | | % | Concen- | End |
| tration | Buffer | Condition | purity | tration | buffer |
| 50 mg/ml | 20 mM | 4° C. | 98.8 | 50 mg/ml | 20 mM His, |
| | His, pH | | | | pH 6.0 |
| | 6.0 | Post- | 95.7 | 50 mg/ml | 20 mM His, |
| | | Lyophilization | | | pH 6.0 |
| | | Post- | 94.2 | 150 mg/ml | 60 mM His, |
| | | Lyophilization | | | 6% maltose, |
| | | | | | pH 6.0 |
| 50 mg/ml | 20 mM | 4° C. | 98.8 | 50 mg/ml | 20 mM His, |
| | His, | | | | 6% maltose, |
| | 6% | | | | pH 6.0 |
| | maltose, | Post- | 98.8 | 50 mg/ml | 20 mM His, |
| | pH 6.0 | Lyophilization | | | 6% maltose, |
| | | | | | pH 6.0 |
| 50 mg/ml | 20 mM | 4° C. | 98.9 | 50 mg/ml | 20 mM His, |
| | His, | | | | 2% maltose, |
| | 2% | | | | pH 6.0 |
| | maltose, | Post- | 98.6 | 150 mg/ml | 60 mM His, |
| | pH 6.0 | Lyophilization | | | 6% maltose, |
| | | | | | pH 6.0 |
| 50 mg/ml | 6.7 mM | 4° C. | 98.4 | 50 mg/ml | 6.7 mM His, |
| | His, | | | | 2% maltose, |
| | 2% | | | | pH 6.0 |
| | maltose, | Post- | 98.3 | 150 mg/ml | 20 mM His, |
| | pH 6.0 | Lyophilization | | | 6% maltose, |
| | | | | | pH 6.0 |
| | | **Post- | 97.4 | 150 mg/ml | 20 mM His, |
| | | Lyophilization | | | 6% maltose, |
| | | | | | pH 6.0 |

**This lyophilizaton was done overnight; otherwise the lyophilizations were done for 5 h
His = histidine

TABLE 11
RET assay of high concentration, lyophilized 150 mg/ml
formulation of CD4-IgG2

| Cd4-IgG2 formulation | IC₅₀ (μg/ml) | IC₉₀ (μg/ml) |
|---|---|---|
| Clinical sample* | 0.32 | 1.03 |
| Clinical sample* | 0.38 | 1.17 |
| 50 mg/ml-150 mg/ml** | 0.35 | 1.12 |

*The clinical sample, assayed in duplicate, consisted of CD4-IgG2 dissolved in PBS buffer at a concentration of 5.0 mg/ml and diluted to 1 μg/ml prior to the RET assay.
**This sample consisted of CD4-IgG2 reconstituted to a concentration of 150 mg/ml in 20 mM histidine, 6% maltose, pH 6.0 after a 5-h lyophilization of a 50 mg/ml solution of CD4-IgG2 in 6.7 mM histidine, 2% maltose, pH 6.0.

Pharmacotoxicological and Pharmacokinetic Evaluation of CD4-IgG2

Comparative pharmacotoxicological and pharmacokinetic studies were conducted with the high concentration formulations 0, 2 and 3 (see Table 3) injected intravenously, intramuscularly and subcutaneously into three groups of rabbits. No mortality was observed during the study nor were clinical signs of toxicity observed in any animal receiving the three formulations of CD4-IgG2 by any of the three routes of administration. The test rabbits weighed 2.1-2.5 kg and the treatment had no effect on body weight for any group.

Visual examination of the ears for irritation after intravenous injection of all of the formulations revealed very slight to well-defined erythema and no edema at the 1-h period, very slight erythema and very slight edema at 24 h, and all sites normal at 72 h, day 8 and day 15. Visual examination of the intramuscular sites for irritation after injection of the formulations revealed no signs of irritation in any animal at any time point with Formulation 0. Very slight erythema was present at 24 h post-dose while very slight edema was visible at 1 and 24 h with Formulation 2. Very slight edema at the 1 h observation period was the only reaction seen with Formulation 3. Visual examination of the subcutaneous sites for irritation in animals receiving Formulation 0 revealed very slight erythema and very slight edema at 1 hour. Well-defined erythema and slight edema was present at 24 h while all sites returned to normal at 72 h post-dose with Formulation 0. Very slight erythema was observed at the 1-h observation with well-defined erythema present at 24 h with Formulation 2. Formulation 3 revealed no signs of erythema or edema during the course of the study.

In general, the irritation seen was minimal and comparable between each formulation and also each route of administration. The occurrence of edema correlated with the volume of test material injected and was completely reversible with the passage of time. Macroscopic evaluation of intensity of Evans Blue dye showed no significant difference when comparisons were made between each of the formulations and their respective control ears. Necropsy of the animals in each of the three formulation groups revealed no significant lesions attributable to test material treatment.

Intravenous administration of formulations 0, 2 and 3 was not associated with microscopic lesions related to the treatment. Intramuscular administration of formulations 0, 2 and 3 was associated with mild to moderate intramuscular edema with minimal evidence of myocyte degeneration and inflammation. Subcutaneous administration of Formulation 0 was associated with mild focal edema and minimal myocyte degeneration whereas subcutaneous injection of formulations 2 and 3 was not associated with any microscopic lesions.

All other documented changes were considered to reflect incidental, degenerative or inflammatory findings that might be encountered in any other similar group of laboratory rabbits. A single intravenous, intramuscular and subcutaneous administration of the formulations of CD4-IgG2 was not associated with any treatment-related, localized gross or microscopic lesions 15 days post-injection. Thus, in conclusion, the three CD4-IgG2 formulations tested were determined to be non-toxic, relatively non-irritating and well tolerated in all routes and dosages evaluated.

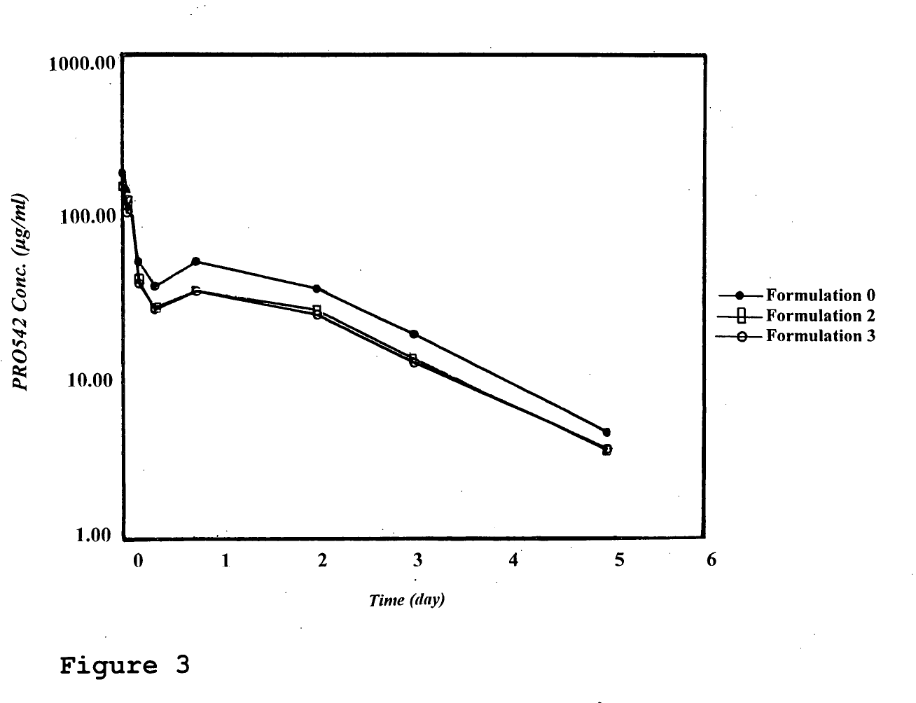

Analysis of sera from rabbits injected with high-concentration formulations of CD4-IgG2 was undertaken to determine the terminal half-life of the therapeutic in vivo. As shown in FIG. 3, CD4-IgG2 administered in formulations 0, 2 and 3 had a long half-life of -1 day which is considerably longer than the serum half-life of 45 minutes of sCD4 in humans following intravenous administration, 9.4 h after intramuscular dosing, and 10.3 h after subcutaneous administration (Schooley et al., 1990; Kahn et al., 1990). A long half-life is advantageous for enhancing the efficacy of the therapeutic, and minimizing the dosage and frequency of administration required to achieve a protective or remedial concentration of the administered drug.

1. A pharmaceutical formulation comprising a CD4-IgG2 chimeric heterotetramer, a histidine buffer and maltose wherein the chimeric heterotetramer is present in said formulation at a concentration between about 25 and about 100 mg/ml, the histidine buffer is present at a concentration between about 3 and about 15 mm, the maltose is present at a concentration between about 1 and about 5%, and said formulation has a ph between about 5.0 and about 7.0.

2. The formulation according to claim 1, wherein the chimeric heterotetramer is present in said formulation at a concentration between about 30 and about 70 mg/ml.

3. The formulation according to claim 2, wherein the chimeric heterotetramer is present at a concentration of about 50 mg/ml.

4. The formulation according to claim 1, wherein the formulation has a pH between about 5.5 and about 6.5.

5. The formulation according to claim 4, wherein the formulation has a pH of about 6.0.

6. canceled

7. The formulation according to claim 1, wherein the histidine buffer is present at a concentration between about 5 and about 10 mM.

8. The formulation according to claim 7, wherein the histidine buffer is present at a concentration of about 6.7 mM.

9. canceled

10. The formulation according to claim 9 claim 1, wherein the maltose is present at a concentration between about 1 and about 3%.

11. The formulation according to claim 10, wherein the maltose is present at a concentration of about 2%.

12. The formulation according to claim 1 further comprising at least one additional anti-viral agent.

13. (canceled)

14. The formulation according to claim 1, wherein the formulation is lyophilized.

15. The formulation according to claim 14, wherein the lyophilized formulation is stable at ambient temperature for at least about twenty-four months.

16. The pharmaceutical formulation according to claim 1, further comprising an amino acid stabilizing agent.

17-26. (canceled)

27. The formulation according to claim 16, wherein the amino acid stabilizing agent is selected from the group consisting of alanine, glycine, proline and glycylglycine, and is present in said formulation at a concentration between about 10 and about 30 mM.

28. (canceled)

29. The formulation according to claim 27, wherein the amino acid stabilizing agent is glycine and the glycine is present in said formulation at a concentration of about 17 mM.

30-33. (canceled)

34. A The pharmaceutical formulation according to claim 1, further comprising a nonionic detergent.

35-38. (canceled)

39. The formulation according to claim 34, wherein the formulation further comprises an amino acid stabilizing agent selected from the group consisting of alanine, glycine, proline and glycylglycine, wherein the amino acid stabilizing agent is present in said formulation at a concentration between about 10 and about 30 mM.

40-54. (canceled)

55. A The pharmaceutical formulation according to claim 1, further comprising at least one osmolality adjusting agent.

56-80. (canceled)

81. A The pharmaceutical formulation according to claim 1, further comprising an amino acid stabilizing agent and at least one osmolality adjusting agent, wherein the amino acid stabilizing agent is selected from the group consisting of alanine, glycine, proline and glycylglycine and is present in said formulation at a concentration between about 10 and about 30 mM, and wherein the at least one osmolality adjusting agent is selected from the group consisting of maltose, trehalose and glycine and is present in the formulation at a concentration e between about 1 and about 4%.

82-83. (canceled)

84. The formulation according to claim 81, wherein the formulation further comprises a nonionic detergent.

85-88. (canceled)

89. The formulation according to claim 81, wherein the formulation is lyophilized.

90. (canceled)

91. A reconstituted, lyophilized pharmaceutical formulation comprising a CD4-IgG2 chimeric heterotetramer, a histidine buffer and maltose wherein the chimeric heterotetramer is present in said formulation at a concentration between about 100 and about 162 mg/ml, the histidine buffer is present at a concentration between about 5 and about 50 mM, the maltose is present at a concentration between about 1 and about 10%, and said formulation has a pH between about 5.0 and about 7.0.

92. The reconstituted formulation according to claim 91, wherein the chimeric heterotetramer is present in said formulation at a concentration e between about 130 and about 155 mg/ml.

93. The reconstituted formulation according to claim 92, wherein the chimeric heterotetramer is present at a concentration of about 150 mg/ml.

94. The reconstituted formulation according to claim 91, wherein the formulation has a pH between about 5.5 and about 6.5.

95. The reconstituted formulation according to claim 94, wherein the formulation has a pH of about 6.0.

96. canceled

97. The reconstituted formulation according to claim 91, wherein the histidine buffer is present at a concentration between about 10 and about 30 mM.

98. The reconstituted formulation according to claim 97, wherein the histidine buffer is present at a concentration of about 20 mM.

99. canceled

100. The reconstituted formulation according to claim 91, wherein the maltose is present at a concentration between about 4 and about 8%.

101. The reconstituted formulation according to claim 100, wherein the maltose is present at a concentration of about 6%.

102. The formulation according to claim 91 further comprising at least one additional anti-viral agent.

103-118. (canceled)

119. The reconstituted formulation according to claim 91, further comprising an amino acid stabilizing agent.

120-191. (canceled)

192. A method of inhibiting infection of a CD4+ cell by a human immunodeficiency virus, which method comprises contacting the human immunodeficiency virus with an amount of a formulation according to claim 91, effective to bind to such human immunodeficiency virus which is in the vicinity of the CD4+ cell, so as to thereby inhibit infection of the CD4+ cell by the virus.

193. A method of preventing CD4+ cells of a subject from becoming infected with human immunodeficiency virus, which method comprises administering to the subject an amount of a formulation according to effective to bind to human immunodeficiency virus present in the subject, so as to thereby prevent the subject's CD4+ cells from becoming infected with human immunodeficiency virus.

194. A method of treating a subject having CD4+ cells infected with human immunodeficiency virus which comprises administering to the subject an amount of a formulation of effective to bind to human immunodeficiency virus present in the subject, so as to thereby treat the subject having CD4+cells infected with human immunodeficiency virus.

195. A method of making a pharmaceutical formulation comprising a CD4-IgG2 chimeric heterotetramer, which method comprises:
(a) dissolving the heterotetramer in a solution comprising a histidine buffer having a concentration between about 3 and about 15 mM and a pH between about 5.0 and about 7.0, maltose at a concentration e between about 1 and about 5%, to produce a first formulation having a concentration of said heterotetramer between about 25 and about 100 mg/ml;
(b) lyophilizing the first formulation to produce a lyophilized formulation; and
(c) adding a diluent to the lyophilized formulation to produce a reconstituted pharmaceutical formulation comprising histidine buffer at a concentration between about 5 and about 50 mM and a pH between about 5.0 and about 7.0, maltose at a concentration between about 1 and about 10%, and containing a concentration of the heterotetramer of between about 100 and about 162 mg/ml.

196-226. (canceled)
